(19) United States
(12) Patent Application Publication
(10) Pub. No.: US 2023/0147312 A1
(43) Pub. Date: May 11, 2023

(54) TREATMENT OF ACUTE RESPIRATORY DISTRESS SYNDROME AND RELATED CONDITIONS WITH ANTAGONISTS OF E-SELECTIN

(71) Applicant: GLYCOMIMETICS, INC., Rockville, MD (US)

(72) Inventors: John L. MAGNANI, Gaithersburg, MD (US); William E. FOGLER, Lutherville-Timonium, MD (US); Armand F. GIRARD, Glenwood, MD (US)

(73) Assignee: GLYCOMIMETICS, INC., Rockville, MD (US)

(21) Appl. No.: 17/907,331

(22) PCT Filed: Mar. 26, 2021
(86) PCT No.: PCT/US2021/024297

§ 371 Date: Sep. 26, 2022

Related U.S. Application Data
(60) Provisional application No. 63/001,185, filed on Mar. 27, 2020.

(51) Int. Cl. A61K 47/55; A61K 31/7068; A61K 45/06; A61K 39/215; A61K 47/54; A61P 31/14; A61P 11/00; A61P 37/06; A61K 31/7032

(52) U.S. Cl. CPC A61K 47/55; A61K 31/7068; A61K 45/06; A61K 39/215; A61K 47/545; A61P 31/14; A61P 11/00; A61P 37/06; A61K 47/549; A61K 31/7032

(57) ABSTRACT

Methods for treating diseases, disorders, and/or conditions associated with COVID-19 comprising administering at least one E-selectin antagonist and/or composition comprising the same are disclosed. Also disclosed are compounds, compositions, and methods for treating patients with acute respiratory distress syndrome.

This application claims the benefit of priority of U.S. Provisional Application No. 63/001,185, filed Mar. 27, 2020, the contents of which are incorporated by reference herein in their entirety.

An early report on the clinical course of 41 hospitalized patients infected with COVID-19 in Wuhan, China was recently published (see Reference 1, infra, which is incorporated by reference in its entirety). Thirteen (32%) were admitted to the ICU, and these patients had higher plasma levels of cytokines, IL-2, IL-7, IL10, GSCF, IP10, MCP1, MIP1A and TNFα. Six of these patients (13%) died, all by progressing to acute respiratory distress syndrome (ARDS).

ARDS is a severe acute inflammatory reaction in the lung and often leads to death. Neutrophils play a central role by extravasating from the bloodstream to enter the lungs, degranulate, and release inflammatory cytokines and reactive oxygen species, resulting in the influx of fluid into the alveoli and blocking the ability to exchange oxygen as depicted in FIGS. 1A and 1B (see Reference 2, infra, which is incorporated by reference in its entirety).

To date, no therapies are in routine clinical use that target the mechanism of ARDS. However, recent encouraging results in the treatment of COVID-19 patients in Italy have been reported using the drug tocilizimab. Tocilizimab is an antibody directed against the cytokine, IL-6 (FIG. 2) and is approved for use as an anti-inflammatory drug. Of six patients in the intensive care unit (ICU), three showed improvement, two stabilized, and one died. Genentech is now launching a Phase III randomized, double-blind, placebo-controlled trial named COVACTA using tocilizamib (Actemira) to treat adults with severe COVID-19 infection.

E-selectin is required for extravasation of neutrophils from the bloodstream to the lungs during ARDS. IL-6 can induce expression of E-selectin (see Reference 3, infra, which is incorporated by reference in its entirety). However, IL-6 is only one of many other cytokines known to induce expression of E-selectin on human endothelial cells. Illustratively, in Reference 4 (infra, which is incorporated by reference in its entirety), human umbilical vein endothelial cells (HUVEC) and human dermal microvascular endothelial cells (HDMEC) were treated with cytokines IL-1β, TNFα, and IL-10 for over 4 hours. Cells in suspension after trypsinization were incubated with an anti-E-selectin antibody, washed, and labeled with a second FITC-labeled goat anti-rat Ab to detect bound antibody to E-selectin. Labeled cells were analyzed for expression of E-selectin by flow cytometry using FACscan. As shown by flow analysis, all three cytokines (FIG. 3), TNFα, IL-1β, and IL-10 stimulate expression of E-selectin compared to the negative control of cells treated only with cell culture medium (Medium). Endothelial cells derived from either umbilical vein (HUVEC, A) or dermal microvasculature (HDMEC, B) both express E-selectin after stimulation by all three cytokines.

Infection by COVID-19 can lead to pneumonia especially in elderly patients. Various biomarkers and clinical scoring methods have been analyzed to differentially diagnose ARDS relative to non-ARDS pneumonia (see Reference 5, infra, which is incorporated by reference in its entirety). As shown in Table 1, while soluble E-selectin (sE-selectin) association with ARDS relative to non-ARDS was strongly significant (P=0.003), the association of IL-6 was not (P=0.145).

TABLE 1
LIPS, APACHE2 score, and levels of ANG-2, sE-selectin, IL-6,
and IL-8 in the ARDS and non-ARDS groups

| | | | test or | |
|---|---|---|---|---|
| | | Non-ARDS | Student's | |
| | ARDS (n = 45) | (n = 113) | t-test | P value |
| ANG-2 (ng/ml) | 7.36 ± 5.99 | 3.05 ± 2.98 | 4.601 | <0.001 |
| IL-8 (pg/ml) | 97.82 ± 188.99 | 73.16 ± 314.70 | 0.491 | 0.624 |
| IL-6 (pg/ml) | 117.54 ± 182.08 | 79.32 ± 132.47 | 1.464 | 0.145 |
| sE-selectin (ng/ml) | 10.61 ± 5.56 | 7.84 ± 5.18 | 2.968 | 0.003 |
| APACHE2 score | 18.5 ± 7.2 | 14.7 ± 6.0 | | 0.001 |
| LIPS | 5.6 ± 1.8 | 4.4 ± 2.1 | | 0.001 |

ANG-2: angiopoietin-2;
IL: interleukin;
LIPS: lung injury prediction score. Data are presented as the mean ± SD or n (%). Analysis performed using West or Student's Ltest.

As pneumonia progresses, a complication known as acute lung injury (ALI) from ARDS may result. This is a severe complication and the main cause of death in COVID-19 patients. The need to identify ALI/ARDS patients who are at high risk prompted a study of potential elevated biomarkers in this population (see Reference 6, infra, which is incorporated by reference in its entirety).

As shown in Table 2 below, by univariate regression analysis, only soluble E-selectin (sEs; sE-selectin) was a significant risk factor (P=0.044) identifying patients with complicating acute lung injury (ALI). All other factors (gender, WBC, CRP, LDH, BUN, Na, glucose, and hematocrit) were not significant.

TABLE 2
Univariate logistic regression analysis for factors identifying complicating
ALI/cALI in patients with pneumonia

| Variable | Coefficient | SD | P value |
|---|---|---|---|
| Age, years | 0.022 | 0.038 | 0.570 |
| Male gender | 0.144 | 0.489 | 0.769 |
| sES, ng/mL | −0.072 | 0.036 | 0.044 |
| WBC, per 1000/μL | −0.059 | 0.103 | 0.565 |
| CRP, mg/dL | −0.113 | 0.067 | 0.091 |
| LDH, IU/L | −0.012 | 0.007 | 0.073 |
| BUN, mg/dL | −0.044 | 0.049 | 0.368 |
| Na, mEq/L | 0.104 | 0.08 | 0.194 |
| Blood glucose, | 0.0003 | 0.01 | 0.977 |
| mg/dL | | | |
| Hematocrit, % | −0.035 | 0.078 | 0.658 |

ALT, acute lung injury;
BUN, blood urea nitrogen;
cALI, clinical status comparable to ALI;
CRP, C-reactive protein;
LDH, lactate dehydrogenase;
sES, soluble E-selectin;
WBC, white blood cell count.

The factors identifying ALI patients were further analyzed by multiple logistic regression analysis and once again the only significant risk factor (P=0.021) for development of ALI/ARDS was identified as soluble E-selectin (sEs), as shown below (Table 3).

TABLE 3
Multiple logistic regression analysis for factors identifying
complicating ALI/cALI in patients with pneumonia

| Variable | OR | 95% CI | | P value |
|---|---|---|---|---|
| sES, per 1 ng/mL increase | 1.099 | 1.012 | 1.260 | 0.021 |
| CRP, per 1 mg/dL increase | 1.029 | 0.829 | 1.293 | 0.795 |
| LDH, per 1 IU/L increase | 1.017 | 0.999 | 1.046 | 0.052 |

ALI, acute lung injury;
cALI, clinical status comparable to ALI;
CI, confidence interval;
CRP, C-reactive protein;
LDH, lactate dehydrogenase;
sES, soluble E-selectin;
OR, odds ratio.

Soluble E-selectin (sEs; sE-selectin) levels were determined by a latex photometric immunoassay (see Reference 5, infra) and was further developed for diagnostic use. Using receiver operating characteristic (ROC) curve analysis, the cutoff value for determining ALI/ARDS was 40.1 ng/ml of sEs with a P value of 0.044 and a sensitivity of 0.8 and a specificity of 0.8 (see Reference 6, ifra, which is incorporated by reference in its entirety) as shown in FIG. 4.

The association between organ failures with increasing levels of sE-selectin [D_{A}N(normal) to D_{A}E(elevated)] in critically ill patients with systemic inflammatory response syndrome (SIRS) was assessed using the latex photometric immunoassay (see Reference 7, infra, which is incorporated by reference in its entirety). The most statistically significant association of increasing levels of sE-selectin with a P value of 0.0001 was respiratory failure (Table 4).

TABLE 4
Prevalence of various organ failures during the
first 5 hospital days in SIRS patients

| | Prevalence (%) | | |
|---|---|---|---|
| | D_{A}N | D_{A}E | |
| Organ failure | (n = 28) | (n = 22) | P values |
| CNS | 32.1 | 54.6 | 0.5054 |
| CVS | 10.7 | 40.9 | 0.0419* |
| Hepatic failure | 0 | 9.1 | 0.1417 |
| Renal failure | 0 | 31.8 | 0.0052* |
| Respiratory failure | 14.3 | 68.2 | 0.0001* |
| Coagulation system failure | 3.5 | 27.3 | 0.0345* |

*P <0.05.

Inflammatory neutrophils in the lung play a major role in ARDS (see Reference 8, infra, which is incorporated by reference in its entirety). Degranulation releases myeloperoxidase (MPO) converting lipids into the bioreactive species, 2-chlorofatty acid (2-CIFA). Two different common molecular species of the bioreactive 2-CIFA are 2-chloropalmitic acid (2-CIPA) and 2-chlorostearic acid (2-CISA).

Plasma concentrations of 2-CIPA and 2-CISA were determined on initial entrance to the ICU for 198 sequential subjects enrolled in the Molecular Epidemiology of Sepsis in the ICU (MESSI) study (see Reference 7, infra). As seen in FIG. 5, both bioreactive lipids (2-CIPA and 2 CISA) from the action of inflammatory neutrophils are associated with strong significance (P<0.001) with the development of ARDS within 6 days of entering the ICU.

Endothelial activation is well-known to occur during ARDS. The analysis of both 2-CIFA species with markers of endothelial activation revealed that significant correlations of both species were only observed with E-selectin, whereas Ang-2 and sThrombomodulin only correlated with 2-CIPA. No correlations were observed with VWF and sICAM-1 (see Table 5).

TABLE 5
Plasma concentrations of 2-CIFA correlate with Ang-2,
E-selectin, and soluble thrombomodulin

| | | vWF | | | |
|---|---|---|---|---|---|
| | Ang-2 | (% | E-selectin | sThrombomodulin | sICAM-1 |
| | (pg/ml) | control) | (ng/ml) | (ng/ml) | (ng/ml) |
| Free | 0.22, | 0.10, | 0.16, | 0.27, | 0.08, |
| 2- | p = | p = | p = | p = | p = |
| CIPA | 0.0056^{A} | 0.1876 | 0.0403^{A} | 000084 | 0.2974 |
| Free | 0.12, | 0.06, | 0.24, | 0.10, | 0.10, |
| 2- | p = | p = | p = | p = | p = |
| CISA | 0.1210 | 0.4320 | 0.0020^{A} | 0.2143 | 0.1968 |

Spearman rank correlation (rho) and P values between plasma endothelial markers and plasma-free 2-CIFA molecular species.
^{A}Significant correlation.
sThrombomodulin, soluble thrombomodulin;
sICAM-1, soluble ICAM-1.

Human lung microvascular endothelial cells (HMVEC-L) were treated with 10 μM 2-CIPA and assayed for adherence of human neutrophils (FIG. 6). The bioreactive 2-CIPA promoted neutrophil adhesion, whereas normal PA (palmitic acid) did not suggest that 2-CIPA from inflammatory neutrophils may act in a positive feedback loop to promote further influx of neutrophils into the lung leading to ARDS.

As described above, the products of degranulating inflammatory neutrophils (chloro fatty acids) stimulate the expression of E-selectin, thereby promoting the influx of more inflammatory neutrophils into the lung. Whether soluble E-selectin from activated endothelium could also be pro-inflammatory was studied in reference 9 (infra, which is incorporated by reference in its entirety). The level of circulating soluble E-selectin was determined using a standard ELISA-based assay on the serum collected from 49 patients determined “at risk” for developing ARDS. As shown in FIG. 7, the level of circulating soluble E-selectin (sE-selectin) is clearly elevated only in ARDS patients.

To determine whether soluble E-selectin itself may be pro-inflammatory, the effects of the addition of recombinant soluble E-selectin (E-zz) on neutrophil functions were examined. Not only did treatment of E-zz cause proinflammatory effects of neutrophil polarization along with activation of neutrophil β2 integrins, E-zz also surprisingly stimulated neutrophil reactive oxidant species production and the release of superoxide anions.

As shown in FIG. 8, soluble recombinant E-selectin (E-zz) and soluble recombinant VCAM (V-zz) were tested for their effects on freshly isolated human neutrophils. Only E-zz was shown to promote neutrophil respiratory burst. An increase in reactive oxygen species was clearly observed using dihydrorhodamine (DHR) as a fluorescent substrate. In addition, neutrophil superoxide release increased over 3-fold as determined by measuring superoxide dismutase—inhibitable cytochrome c reduction. These effects were specifically induced by E-zz and not V-zz. Furthermore, they could be totally inhibited by the addition of an F(ab)2 fragment of an anti-E-selectin antibody (ENA2).

These pro-inflammatory effects of soluble E-selectin suggest that E-selectin is a target for therapy not only on the endothelial cell surface but also when it is enzymatically released into the bloodstream as soluble E-selectin. Interestingly, as a functional biomarker of ARDS, it is not elevated over non-acute lung injury, but the blood levels of circulating soluble E-selectin (sES) decrease as ARDS, resolves further supporting it as a therapeutic target for ARDS. (See Reference 6; FIG. 9).

Support for the use of glycomimetic antagonists to treat COVID-19 patients with respiratory symptoms comes from some early studies using the native carbohydrate ligand that bind E-selectin known as sialyl Le^{x} (SLX) (see Reference 10, infra, which is incorporated by reference in its entirety). A model of acute lung injury in rats was developed by intratracheal deposition of anti-BSA IgG antibodies in the lungs followed by intravenous injection of BSA. This model of acute lung injury is characterized by an influx of neutrophils with extensive intraalveolar hemorrhage similar to that observed in ARDS.

Rats in this model were treated by intravenous injection of sialyl Le^{x} (SLX) tetrasaccharide or sialyl Le^{x} (SLX) pentasaccharide intravenously at 2.5, 3.0, and 3.5 hours after injection of BSA. The effects of treatment with both of these natural carbohydrate ligands that block E-selectin are shown in FIG. 10. The influx of neutrophils was significantly inhibited as determined by the specific neutrophil marker of myeloperoxidase (MPO). Both hemorrhage and permeability indices were significantly inhibited as well. These finding were well supported by immunohistology of lung tissue.

Glycomimetic compounds are small molecules that are rationally designed to mimic the bioactive conformation of functional carbohydrates but with increased binding affinity and improved drug-like properties. Three glycomimetic compounds invented and synthesized at GlycoMimetics, Inc. (GMI) have been tested in human clinical trials. Two of these compounds, Rivipansel (GMI-1070) and Uproleselan (GMI-1271), have passed Phase 2 clinical trials. Both compounds are potent antagonists of E-selectin and have demonstrated no measurable toxicities in animals or in Phase 1 clinical trials.

As stated above, circulating soluble E-selectin levels are significantly elevated in ARDS patients and subside as the crisis resolves. Soluble E-selectin itself has pro-inflammatory effects on neutrophils and its reduction should have therapeutic effects. FIG. 11 is from a clinical trial testing the effects of GMI-1070 on vaso-occlusive crisis in sickle cell patients (see Reference 11, infra, which is incorporated by reference in its entirety). Prior to dosing GMI-1070, these patients have elevated levels of circulating soluble E-selectin. In patients treated with GMI-1070, the plasma concentration of soluble-E-selectin (sE-Sel) is reduced.

A different glycomimetic antagonist of E-selectin known as GMI-1271 was designed for increased specificity for E-selectin to be used in the treatment of acute myelogenous leukemia (AML) patients. In a Phase ½ clinical trial (see Reference 11, infra), AML patients express high levels of sE-selectin in their plasma. After treatment with GMI-1271, sE-selectin circulating levels in all patients were significantly (P<0.0001) reduced (see Reference 12, infra, which is incorporated by reference in its entirety; FIG. 12).

In some embodiments of the present disclosure, antagonists of E-selectin which include glycomimetic antagonists (such as, e.g., GMI-1070 and GMI-1271) may be used to treat COVID-19 patients with respiratory symptoms that may lead to ARDS or who have already been diagnosed with ARDS.

Both of GMI's glycomimetic antagonists of E-selectin that have been tested through Phase 2 clinical trials (GMI-1070 and GMI-1271) are at least 500 to 1000-fold more potent in binding and inhibiting E-selectin than the natural carbohydrate ligands used in this model. In addition, both of these glycomimetic drugs possess far better drug-like properties and have shown no toxicity even at relatively high concentrations in animals or humans.

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description and attached drawings.

### BRIEF DESCRIPTION OF THE DRAWINGS

FIG. 1A is a diagram illustrating initiation of ARDS: neutrophil extravasation by E-selectin.

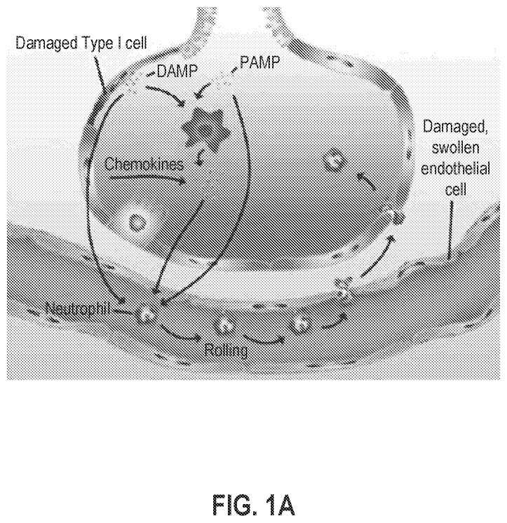

FIG. 1B is a diagram illustrating progression resulting in vascular leakage.

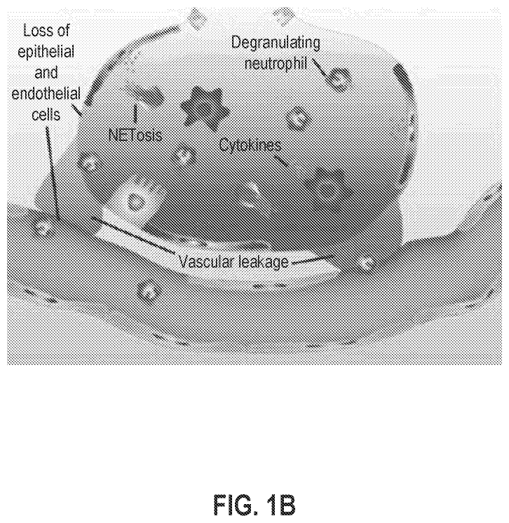

FIG. 2 is a diagram illustrating tocilizimab, an antibody directed against the cytokine, IL-6.

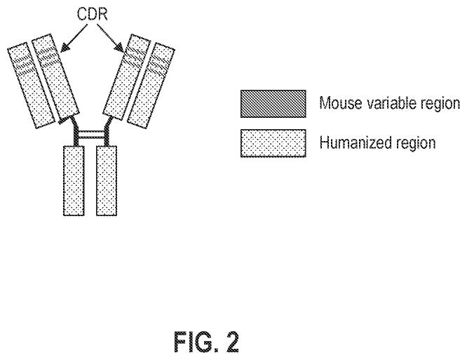

FIG. 3 is a comparison of the induction of E-selectin by IL-1β, TNF-α, and IL-10 in HDMEC (A) and HUVEC (B) cells.

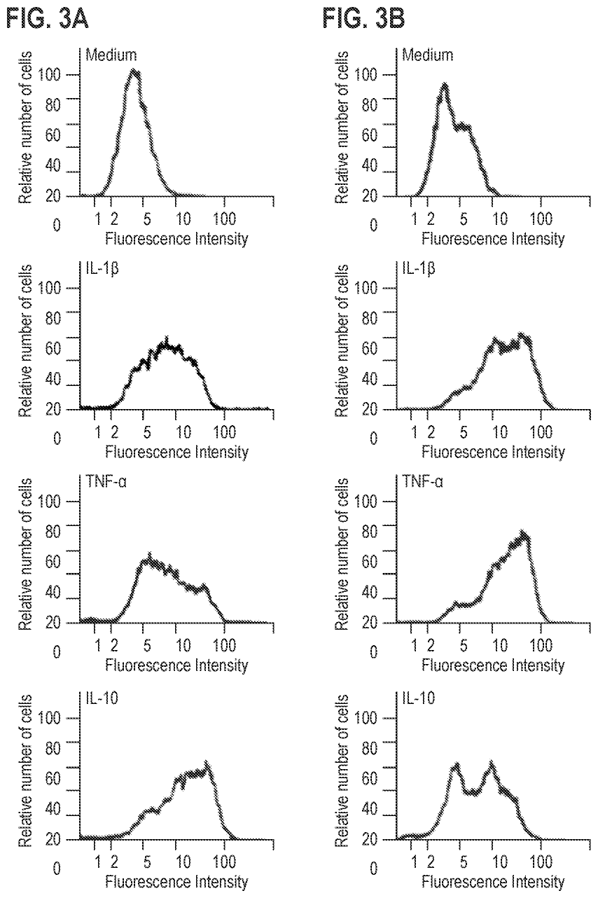

FIG. 4 is a graph illustrating the determination of the soluble E-selectin (sE-selectin; sES) cut-off value for discrimination of complicating ALI/cALI in pneumonia patients.

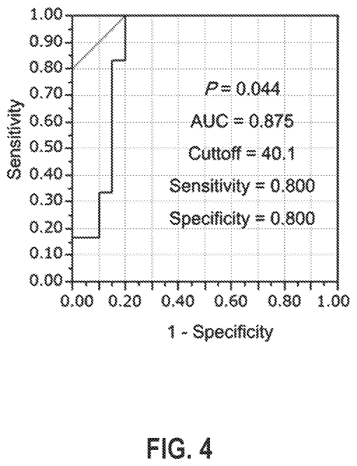

FIG. 5 is a graph illustrating that septic patients with ARDS display higher day 0 plasma concentrations of free 2-CIFAs. Compared with subjects who never developed ARDS, those who developed ARDS within the first 6 days of sepsis have higher circulating levels of free 2-CIPA and free 2-CISA on the day of ICU admission (compared by the Wilcoxon rank-sum test). All values were included in the analyses; however, outliers higher than the y axis are not shown. Box plots show median and 25th and 75th percentile, and whiskers show minima and maxima for each condition. For the ARDS group, 9 and 11 outliers had values above 4 nM 2-CIPA or above 8 nM 2-CISA, respectively. In the non-ARDS group, there were 2 outliers with values above 8 nM 2-CISA, but none above 4 nM 2-CIPA.

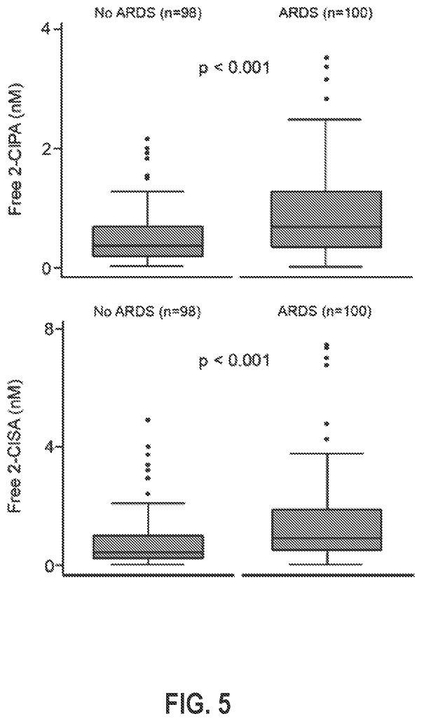

FIG. 6 is a graph illustrating that, in comparison to palmitic acid (PA), exogenously added 2-CIPA increases adhesion of neutrophils to HMVEC-L.

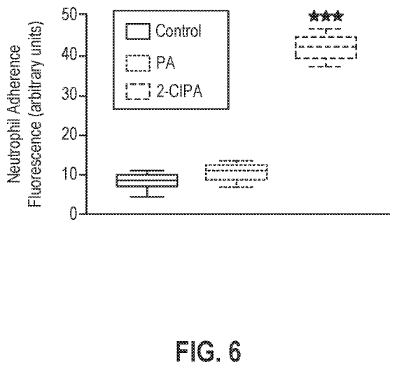

FIG. 7 is a graph illustrating that soluble E-selectin (sE-selectin) is elevated in patients with established ARDS. Soluble E-selectin was quantified in serum from 49 patients at risk of ARDS (perforated bowel, non-ARDS progression or ARDS progression) and 10 patients who had established ARDS, using a sandwich ELISA.

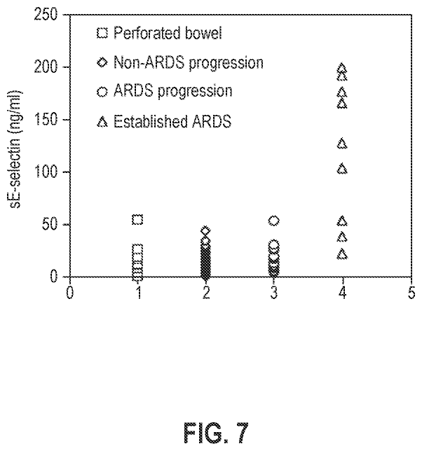

FIG. 8 is a graph illustrating the effect of recombinant soluble E-selectin (E-zz) upon neutrophil respiratory burst. Freshly isolated neutrophils were pre-incubated with E-zz or recombinant soluble VCAM (V-zz) (2.5 μg/ml) for 15 minutes at 20° C. in HBSS. (A) DHR was then added at 1 μM and cells were incubated at 37° C. for 5 minutes. After 15 minutes at 37° C. in a shaking water-bath, DHR oxidation was analyzed by flow cytometry. Medium: HBSS alone. (B) Superoxide anion release measurement. After incubation with E-zz or V-zz (2.5 μg/ml) in the presence of cytochrome c, the superoxide dismutase inhibitable reduction of cytochrome c was then determined by measuring the peak absorbance between 535 and 565 nm using a Pye-Unicam scanning spectrophotometer.

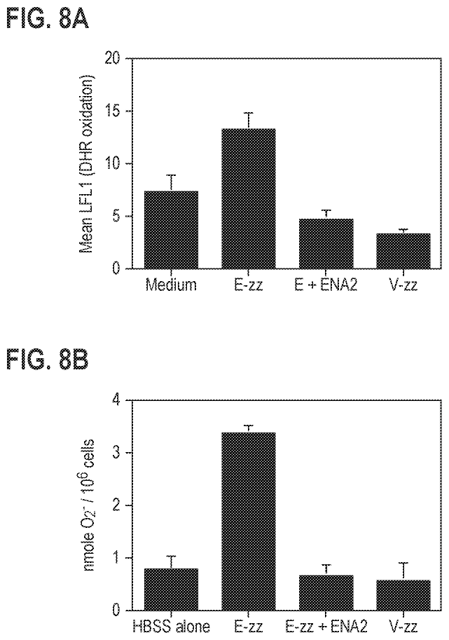

FIG. 9 is a diagram illustrating the time course of soluble E-selectin (sES) according to complicating acute lung injury (ALI)/clinical status comparable to ALI (cALI). sES levels were higher in pneumonia patients with ALI/cALI than in those without ALI/cALI. sES levels decreased after commencement of treatment in the ALI/cALI group (P=0.017). However, sES levels in the non-ALI/cALI group were not significantly different at each time point (P=0.075).

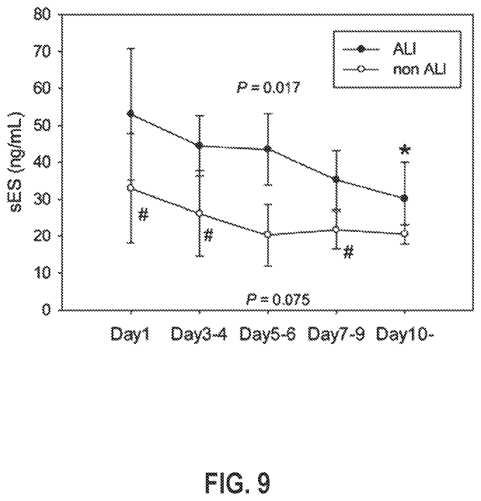

FIG. 10 is a diagram illustrating the ability of tetrasaccharide and pentasaccharide (glycoside) derivatives of sialyl Le^{x} (SLX) to reduce lung vascular injury after intrapulmonary deposition of IgG immune complexes. Oligosaccharides were infused intravenously in the indicated doses at 2.5, 3.0, and 3.5 h after intrapulmonary deposition of IgG immune complexes. Positive control values (PBS-treated rats) for permeability (A), hemorrhage (B), and MPO (C) parameters were 0.94±0.01, 0.31±0.01, and 0.64±0.02, respectively, and are shown by the stippled horizontal bar.

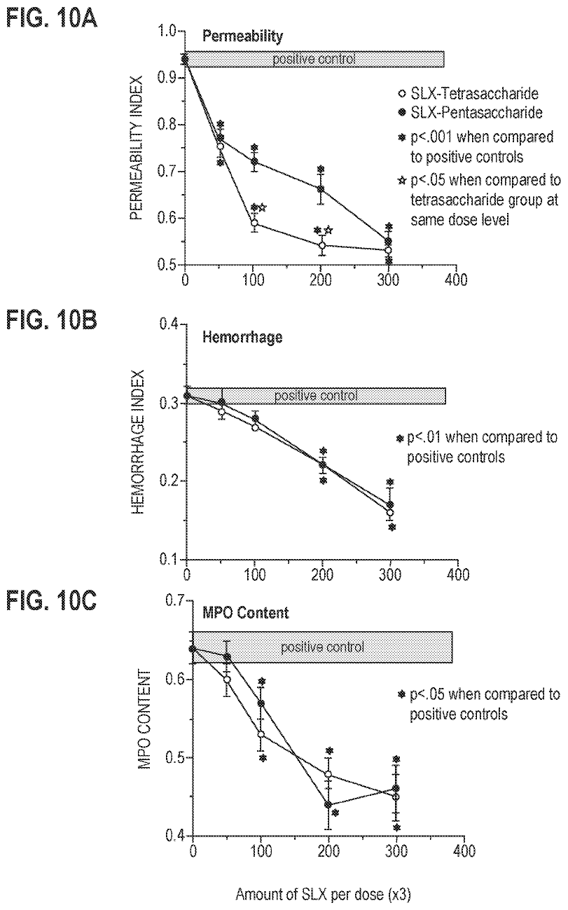

FIG. 11 is a diagram illustrating that pan-selectin antagonist GMI-1070 reduces soluble E-selectin levels while improving clinical outcomes in SCD vaso-occlusive crises.

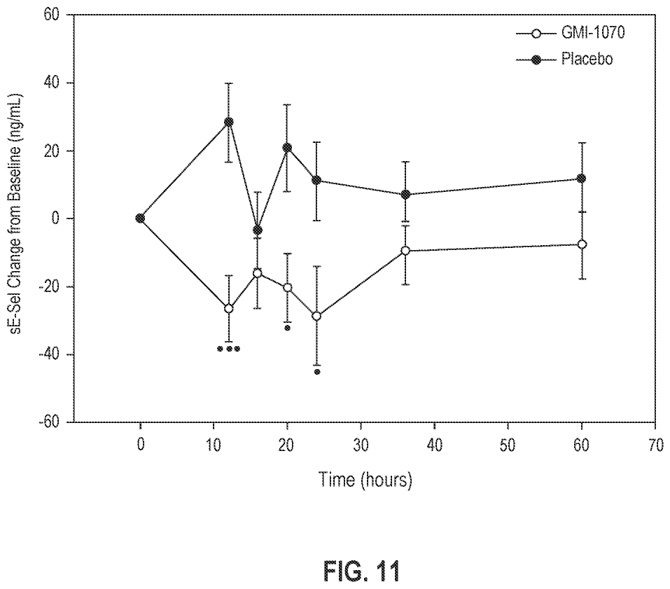

FIG. 12 is a diagram illustrating that plasma soluble E-selectin levels decreased over the treatment period in all dose groups. This response was seen both in mean levels and Area-Under-Effect-Curve (AUEC). Decrease from baseline to Day 8 (at the end of 6 days of GMI-1271 given concurrently with MEC chemotherapy) was highly significant (p<0.0001) with no dose response.

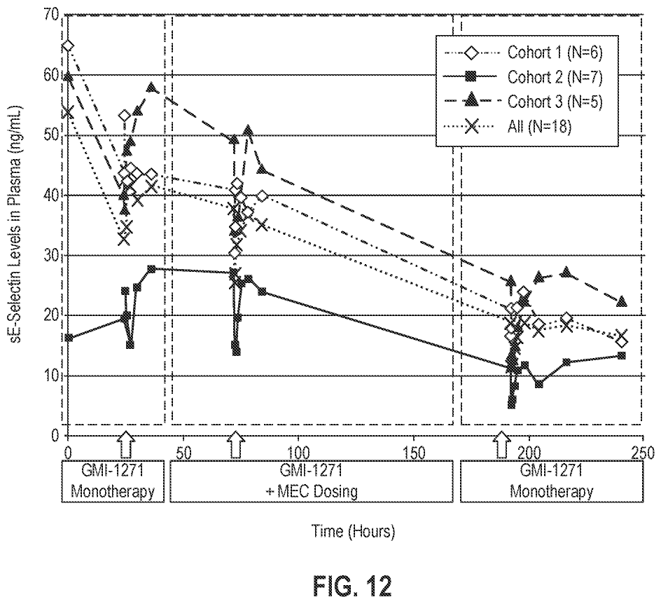

### DEFINITIONS

As used herein, “a” or “an” entity refers to one or more of that entity, e.g., “a compound” refers to one or more compounds or at least one compound unless stated otherwise. As such, the terms “a” (or “an”), “one or more”, and “at least one” are used interchangeably herein.

As used herein, “administration” of a compound to a patient refers to any route (e.g., oral delivery) of introducing or delivering the API to the patient. Administration includes self-administration and the administration by another.

As used herein, an “effective amount” or “effective dose” refers to an amount of a compound that treats, upon single or multiple dose administration, a patient suffering from a condition. An effective amount can be determined by the attending diagnostician through the use of known techniques and by observing results obtained under analogous circumstances. In determining the effective amount, a number of factors are considered by the attending diagnostician, including, but not limited to: the patient's size, age, and general health; the specific condition, disorder, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances.

As used herein, the term “E-selectin antagonist” includes antagonists of E-selectin only, as well as antagonists of E-selectin and either P-selectin or L-selectin, and antagonists of E-selectin, P-selectin, and L-selectin. The terms “E-selectin antagonist” and “E-selectin inhibitor” are used interchangeably herein.

E-selectin antagonists include the glycomimetic compounds described herein. E-selectin antagonists also include antibodies, polypeptides, peptides, peptidomimetics, and aptamers which bind at or near the binding site on E-selectin to inhibit E-selectin interaction with sialyl Le^{a}(sLe^{a}) or sialyl Le^{x} (sLe^{x}).

Further disclosure regarding E-selectin antagonists suitable for the disclosed methods (e.g., compounds and compositions) may be found in U.S. Pat. No. 9,254,322, issued Feb. 9, 2016, and U.S. Pat. No. 9,486,497, issued Nov. 8, 2016, which are hereby incorporated by reference. In some embodiments, the E-selectin antagonist is chosen from E-selectin antagonists disclosed in U.S. Pat. No. 9,109,002, issued Aug. 18, 2015, which is hereby incorporated by reference. In some embodiments, the E-selectin antagonist is chosen from heterobifunctional antagonists disclosed in U.S. Pat. No. 8,410,066, issued Apr. 2, 2013, and U.S. Pat. No. 10,519,181, issued Dec. 31, 2019, which are hereby incorporated by reference. Further disclosure regarding E-selectin antagonists suitable for the disclosed methods and compounds may be found in U.S. Publication No. US2019/0233458, published Aug. 1, 2019, WO2019/133878, published Jul. 4, 2019, WO 2020/139962, published Jul. 2, 2020, WO 2020/219419, published Oct. 29, 2020, and WO 2020/219417, published Oct. 29, 2020, which are hereby incorporated by reference.

As used herein, an amount expressed in terms of “mg of at least one compound chosen from [X] and pharmaceutically acceptable salts thereof” is based on the total weight of the free base of [X] present, in the form of the free base and/or one or more pharmaceutically acceptable salts of [X].

As used herein, the term “increase” refers to altering positively by at least 1%, including, but not limited to, altering positively by at least 5% (e.g., by 5%), altering positively by at least 10% (e.g., 10%), altering positively by at least 25% (e.g., by 25%), altering positively by at least 30% (e.g., by 30%), altering positively by at least 50% (e.g., by 50%), altering positively by at least 75% (e.g., by 75%), or altering positively by 100%, altering positively by 5% to 10%, altering positively by 5% to 15%, altering positively by 5% to 25%, etc.

As used herein, the term “modulate” refers to altering positively or negatively. Non-limiting example modulations include an at least 1% (e.g., a 1%) change, an at least a 2% (e.g., 2%) change, an at least a 5% (e.g., 5%) change, an at least a 10% (e.g., a 10%) change, an at least a 25% (e.g., 25%) change, an at least a 50% (e.g., 50%) change, an at least a 75% (e.g., a 75%) change, a 100% change, a 5% to 10% change, a 5% to 15% change, a 5% to 25% change, etc.

As used herein, the terms “patient” and “subject” are used interchangeably and refer to a human.

As used herein, a “pharmaceutically acceptable excipient” refers to a carrier or an excipient that is useful in preparing a pharmaceutical composition. For example, a pharmaceutically acceptable excipient is generally safe and includes carriers and excipients that are generally considered acceptable for mammalian pharmaceutical use. As a non-limiting example, pharmaceutically acceptable excipients may be solid, semi-solid, or liquid materials which in the aggregate can serve as a vehicle or medium for the active ingredient. Some examples of pharmaceutically acceptable excipients are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

As used herein, the term “pharmaceutically acceptable salts” includes sodium, potassium, lithium, ammonium (substituted and unsubstituted), calcium, magnesium, iron, zinc, copper, manganese, and aluminum salts. Pharmaceutically acceptable salts may, for example, be obtained using standard procedures well known in the field of pharmaceuticals.

As used herein, the term “reduce” refers to altering negatively by at least 1% including, but not limited to, altering negatively by at least 5% (e.g., by 5%), altering negatively by at least 10% (e.g., by 10%), altering negatively by at least 25% (e.g., by 25%), altering negatively by at least 30% (e.g., by 30%), altering negatively by at least 50% (e.g., by 50%), altering negatively by at least 75% (e.g., by 75%), altering negatively by 100%, altering negatively by 5% to 10%, altering negatively by 5% to 15%, altering negatively by 5% to 25%, etc.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art.

### Embodiments

Without limitation, some embodiments of the disclosure include:

1. A method of treating COVID-19 in a patient in need thereof, the method comprising administering to the patient an effective amount of at least one E-selectin antagonist or a pharmaceutical composition comprising an effective amount of at least one E-selectin antagonist.
2. A method of treating a disease, disorder, and/or condition associated with COVID-19 in a patient in need thereof, the method comprising administering to the patient an effective amount of at least one E-selectin antagonist or a pharmaceutical composition comprising an effective amount of at least one E-selectin antagonist.
3. The method according to Embodiment 2, wherein the disease, disorder, and/or condition associated with COVID-19 is acute lung injury.
4. The method according to Embodiment 2, wherein the disease, disorder, and/or condition associated with COVID-19 is acute respiratory distress syndrome.
5. The method according to Embodiment 2, wherein the disease, disorder, and/or condition associated with COVID-19 is pneumonia.
6. The method according to Embodiment 2, wherein the disease, disorder, and/or condition associated with COVID-19 is ARDS pneumonia.
7. A method of treating acute respiratory distress syndrome in a patient in need thereof, the method comprising administering to the patient an effective amount of at least one E-selectin antagonist or a pharmaceutical composition comprising an effective amount of at least one E-selectin antagonist.
8. A method of treating pneumonia in a patient in need thereof, the method comprising administering to the patient an effective amount of at least one E-selectin antagonist or a pharmaceutical composition comprising an effective amount of at least one E-selectin antagonist.
9. The method according to any one of Embodiments 1 to 8, wherein the at least one E-selectin antagonist is chosen from compounds of Formula (I), (Ia), (II), (IIa), (III), (IIIa), (IV), (V), (IVa/Va), (IVb/Vb), (VI), (VII), and (VIII) and pharmaceutically acceptable salts of any of the foregoing.
10. The method according to any one of Embodiments 1 to 8, wherein the at least one E-selectin antagonist is chosen from compounds of Formula (I):

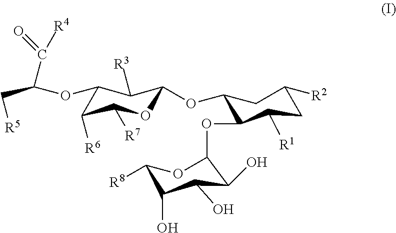

isomers of Formula (I), tautomers of Formula (I), and pharmaceutically acceptable salts of any of the foregoing, wherein:
R¹ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R² is chosen from H, -M, and -L-M;
R³ is chosen from —OH, —NH₂, —OC(═O)Y¹, —NHC(═O)Y¹, and —NHC(═O)NHY¹ groups, wherein Y¹ is chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups;
R⁴ is chosen from —OH and —NZ¹Z² groups, wherein Z¹ and Z², which may be identical or different, are each independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Z¹ and Z² may together form a ring;
R⁵ is chosen from C₃₋₈ cycloalkyl groups;
R⁶ is chosen from —OH, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R⁷ is chosen from —CH₂OH, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R⁸ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
L is chosen from linker groups; and
M is a non-glycomimetic moiety chosen from polyethylene glycol, thiazolyl, chromenyl, —C(═O)NH(CH₂)₁₋₄NH₂, C₁₋₈ alkyl, and —C(═O)OY groups, wherein Y is chosen from C₁₋₄ alkyl, C₂₋₄ alkenyl, and C₂₋₄ alkynyl groups.
   11. The method according to Embodiment 10, wherein the at least one E-selectin antagonist is chosen from compounds of Formula (I), wherein the non-glycomimetic moiety comprises polyethylene glycol.
   12. The method according to Embodiment 10 or 11, wherein L is —C(═O)NH(CH₂)₁₋₄ NHC(═O)—.
   13. The method according to any one of Embodiments 1 to 8, wherein the at least one E-selectin antagonist is chosen from compounds of Formula (Ia):

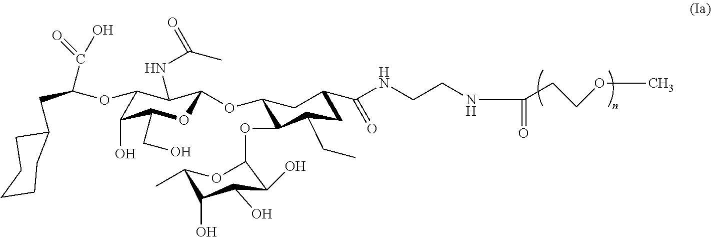

and pharmaceutically acceptable salts thereof, wherein n is chosen from integers ranging from 1 to 100.
14. The method according to Embodiment 13, wherein n is chosen from 4, 8, 12, 16, 20, 24, and 28.
15. The method according to any one of Embodiments 1 to 8, wherein the at least one E-selectin antagonist is chosen from Compound A:

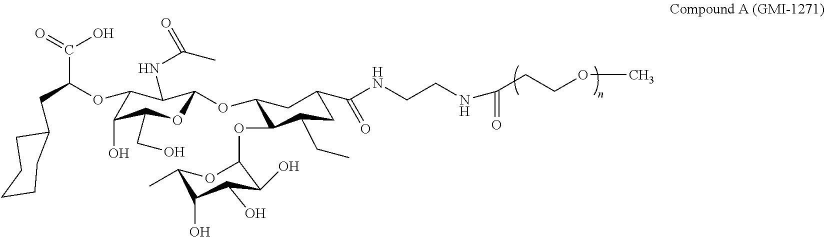

and pharmaceutically acceptable salts thereof.
16. The method according to any one of Embodiments 1 to 8, wherein the at least one E-selectin antagonist is a heterobifunctional antagonist chosen from compounds of Formula (II):

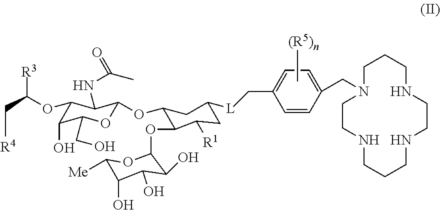

isomers of Formula (II), tautomers of Formula (II), and pharmaceutically acceptable salts of any of the foregoing, wherein:
R¹ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R² is chosen from —OH, —NH₂, —OC(═O)Y¹, —NHC(═O)Y¹, and —NHC(═O)NHY¹ groups, wherein Y¹ is chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups;
R³ is chosen from —CN, —CH₂CN, and —C(═O)Y² groups, wherein Y² is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Z¹ and Z² may together form a ring;
R⁴ is chosen from C₃₋₈ cycloalkyl groups;
R⁵ is independently chosen from H, halo, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
n is chosen from integers ranging from 1 to 4; and
L is chosen from linker groups.
   17. The method according to Embodiment 16, wherein the at least one E-selectin antagonist is a heterobifunctional antagonist chosen from compounds of Formula (IIa):

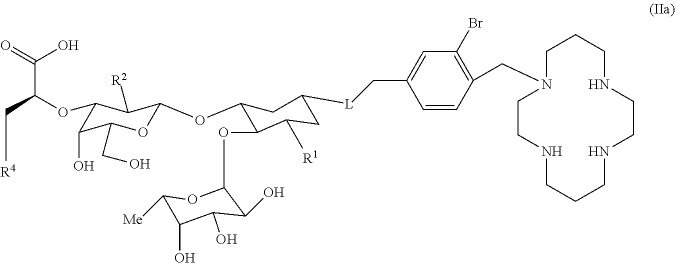

and pharmaceutically acceptable salts thereof.
18. The method according to any one of Embodiments 1 to 8, wherein the at least one E-selectin antagonist is a heterobifunctional antagonist chosen from Compound B:

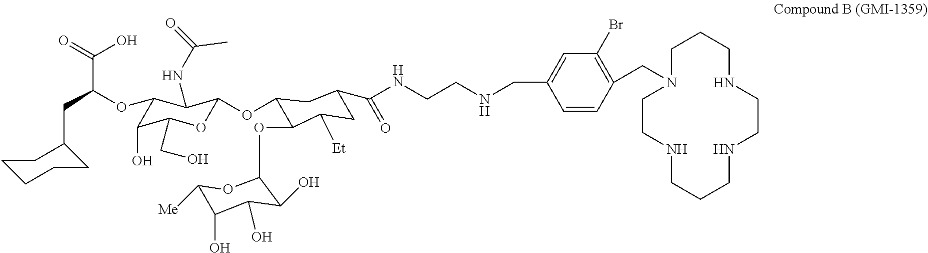 [SMILES: CCC1CC(C(=O)NCCNCc2ccc(CN3CCCNCCNCCCNCC3)c(Br)c2)CC(OC2OC(CO)C(O)C(O[C@@H](CC3CCCCC3)C(=O)O)C2NC(C)=O)C1OC1OC(C)C(O)C(O)C1O]

and pharmaceutically acceptable salts thereof.
19. The method according to any one of Embodiments 1 to 8, wherein the at least one E-selectin antagonist is a heterobifunctional pan-selectin antagonist chosen from compounds of Formula (III):

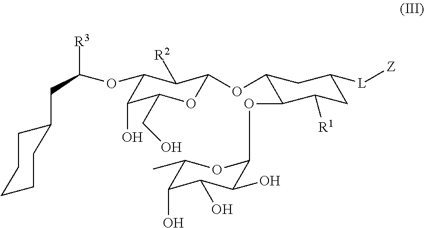

isomers of Formula (III), tautomers of Formula (III), and pharmaceutically acceptable salts of any of the foregoing, wherein:
R¹ is chosen from H, C₁₋₈ alkyl, C₂₋₈ alkenyl, C₂₋₈ alkynyl, C₄₋₁₆cycloalkylalkyl

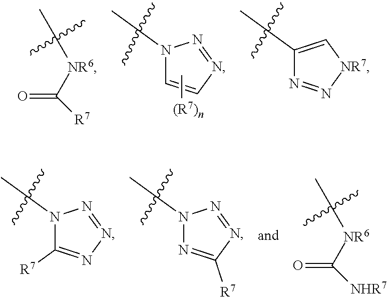

groups;
R² is chosen from C₁₋₈ alkyl, C₂₋₈ alkenyl, C₂₋₈ alkynyl, C₄₋₁₆cycloalkylalkyl, —OH, —OX¹, halo, —NH₂, —OC(═O)X¹, —NHC(═O)X¹, and —NHC(═O)NHX¹ groups, wherein X¹ is chosen from C₁₋₈ alkyl, C₂₋₈ alkenyl, C₂₋₈ alkynyl, C₄₋₁₆cycloalkylalkyl, C₂₋₁₂ heterocyclyl, C₆₋₁₈ aryl, and C₁₋₁₃ heteroaryl groups;
R³ is chosen from —CN, —CH₂CN, and —C(═O)X² groups, wherein X² is chosen from C₁₋₈ alkyl, C₂₋₈ alkenyl, C₂₋₈ alkynyl, —OY², —NHOH, —NHOCH₃, —NHCN, and —NY²Y³ groups, wherein Y² and Y³, which may be identical or different, are independently chosen from H, C₁₋₈ alkyl, C₂₋₈ alkenyl, C₂₋₈ alkynyl, and C₄₋₁₆ cycloalkylalkyl groups, wherein Y² and Y³ may join together to form a ring;
R⁶ is chosen from H, C₁₋₈ alkyl, C₂₋₈ alkenyl, C₂₋₈ alkynyl, C₄₋₁₆ cycloalkylalkyl, and —C(═O)R⁷ groups;
each R⁷ is independently chosen from H, C₁₋₈ alkyl, C₂₋₈ alkenyl, C₂₋₈ alkynyl, C₄₋₁₆ cycloalkylalkyl,

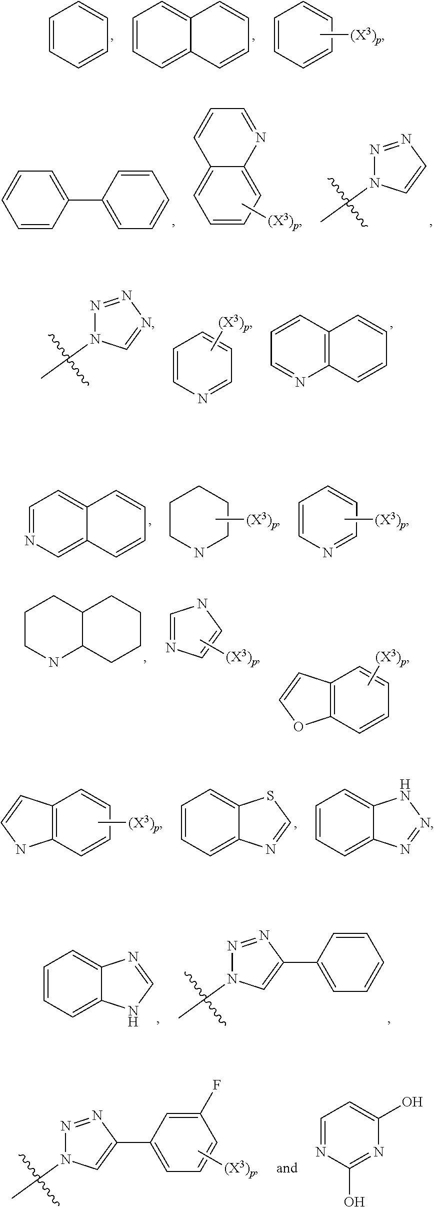

groups, wherein each X³ is independently chosen from H, —OH, Cl, F, N₃, —NH₂, C₁₋₈ alkyl, C₂₋₈ alkenyl, C₂₋₈ alkynyl, C₆₋₁₄ aryl, —OC₁₋₈ alkyl, —OC₂₋₈ alkenyl, —OC₂₋₈ alkynyl, and —OC₆₋₁₄ aryl groups, wherein any of the above ring compounds may be substituted with one to three groups independently chosen from Cl, F, C₁₋₈ alkyl, C₂₋₈ alkenyl, C₂₋₈ alkynyl, C₆₋₁₄ aryl, and —OY⁴ groups, wherein Y⁴ is chosen from H, C₁₋₈ alkyl, C₂₋₈ alkenyl, C₂₋₈ alkynyl, and C₆₋₁₄ aryl groups;
n is chosen from integers ranging from 0 to 2;
p is chosen from integers ranging from 0 to 3;
L is chosen from linker groups; and
Z is chosen from benzyl amino sulfonic acid groups.
   20. The method according to Embodiment 19, wherein the at least one E-selectin antagonist is chosen from Formula (IIIa):

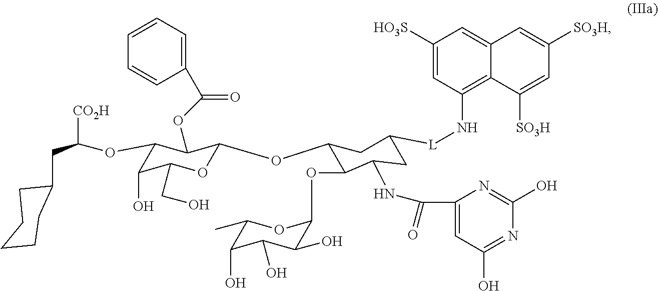

tautomers of Formula (IIIa), and pharmaceutically acceptable salts of any of the foregoing.
21. The method according to any one of Embodiments 1 to 8, wherein the at least one E-selectin antagonist is a heterobifunctional pan-selectin antagonist chosen from Compound C:

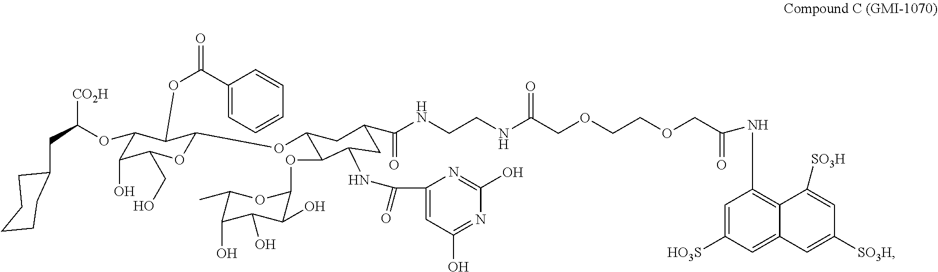 [SMILES: CC1OC(OC2C(NC(=O)c3cc(O)nc(O)n3)CC(C(=O)NCCNC(=O)COCCOCC(=O)Nc3cc(S(=O)(=O)O)cc4cc(S(=O)(=O)O)cc(S(=O)(=O)O)c34)CC2OC2OC(CO)C(O)C(O[C@@H](CC3CCCCC3)C(=O)O)C2OC(=O)c2ccccc2)C(O)C(O)C1O]

tautomers of Compound C, and pharmaceutically acceptable salts of any of the foregoing.
22. The method according to any one of Embodiments 1 to 8, wherein the at least one E-selectin antagonist is chosen from compounds of Formula (IV):

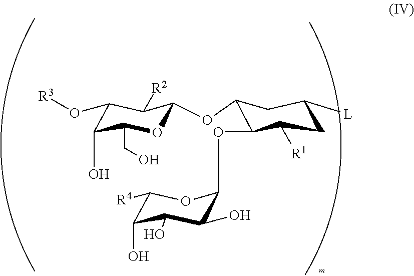

prodrugs of Formula (IV), isomers of Formula (IV), tautomers of Formula (IV), and pharmaceutically acceptable salts of any of the foregoing, wherein
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NY¹Y² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;
each R³, which may be identical or different, is independently chosen from

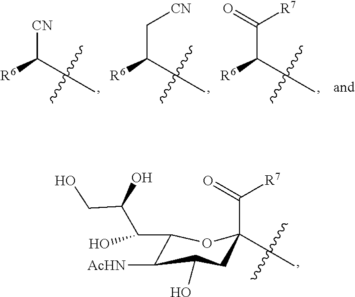

wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is chosen from integers ranging from 2 to 256; and
L is chosen from linker groups.
   23. The method according to any one of Embodiments 1 to 8, wherein the at least one E-selectin antagonist is chosen from compounds of Formula (V):

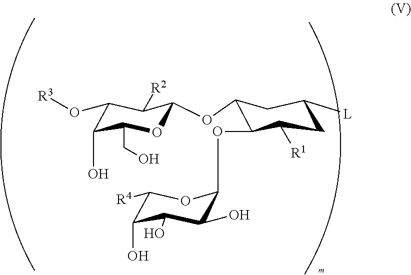

prodrugs of Formula (V), isomers of Formula (V), tautomers of Formula (V), and pharmaceutically acceptable salts of any of the foregoing, wherein
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NY¹Y² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;
each R³, which may be identical or different, is independently chosen from

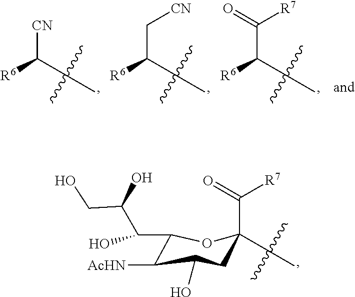

wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³; Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is 2; and
L is chosen from

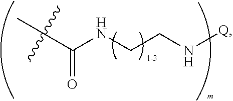

wherein Q is a chosen from

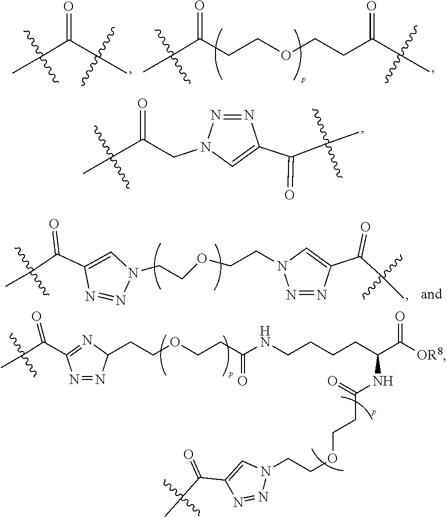

wherein R⁸ is chosen from H, C₁₋₈ alkyl, C₆₋₁₈ aryl, C₇₋₁₉ arylalkyl, and C₁₋₁₃ heteroaryl groups and each p, which may be identical or different, is independently chosen from integers ranging from 0 to 250.
   24. The method according to any one of Embodiments 1 to 8, wherein the at least one E-selectin antagonist is chosen from Compound D:

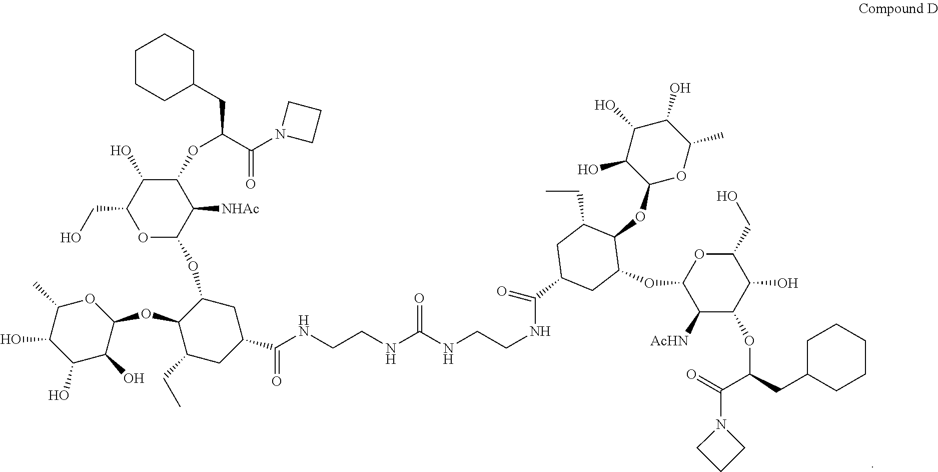 [SMILES: CC[C@H]1C[C@@H](C(=O)NCCNC(=O)NCCNC(=O)[C@@H]2C[C@H](CC)[C@@H](O[C@@H]3O[C@@H](C)[C@@H](O)[C@@H](O)[C@@H]3O)[C@H](O[C@@H]3O[C@H](CO)[C@H](O)[C@H](O[C@@H](CC4CCCCC4)C(=O)N4CCC4)[C@H]3NC(C)=O)C2)C[C@@H](O[C@@H]2O[C@H](CO)[C@H](O)[C@H](O[C@@H](CC3CCCCC3)C(=O)N3CCC3)[C@H]2NC(C)=O)[C@@H]1O[C@@H]1O[C@@H](C)[C@@H](O)[C@@H](O)[C@@H]1O]

25. The method according to any one of Embodiments 1 to 8, wherein the at least one E-selectin antagonist is chosen from Compound E:

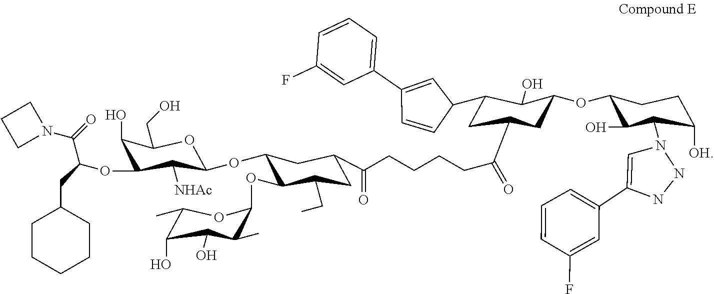

26. The method according to any one of Embodiments 1 to 25, further comprising administering at least one additional therapeutic agent.
27. The method according to Embodiment 26, wherein the at least one additional therapeutic agent is chosen from antiviral and antiretroviral drugs.
28. The method according to Embodiment 26, wherein the at least one additional therapeutic agent is molnupiravir.
29. The method according to Embodiment 26, wherein the at least one additional therapeutic agent is chosen from anti-protozoal agents.
30. The method according to Embodiment 26, wherein the at least one additional therapeutic agent is chosen from IL-6 inhibitors.
31. The method according to any one of Embodiments 1 to 30, further comprising administering at least one COVID-19 vaccine.
32. The method according to any one of Embodiments 1 to 31, wherein the patient is intravenously administered the at least one E-selectin antagonist.
33. The method according to any one of Embodiments 1 to 32, wherein the administration begins within 48 hours of administration of supplemental oxygen to the patient.
34. The method according to any one of Embodiments 10 to 15, wherein the patient is administered a dose in the range of 5 mg/kg to 100 mg/kg of the at least one E-selectin antagonist.
35. The method according to any one of Embodiments 10 to 15, wherein the patient is administered a dose in the range of 5 mg/kg to 100 mg/kg of the at least one E-selectin antagonist once a day.
36. The method according to any one of Embodiments 10 to 15, wherein the patient is administered a dose in the range of 5 mg/kg to 100 mg/kg of the at least one E-selectin antagonist twice a day.
37. The method according to any one of Embodiments 10 to 15, wherein the patient is administered a dose in the range of 5 mg/kg to 100 mg/kg of the at least one E-selectin antagonist for 7 days.
38. The method according to any one of Embodiments 10 to 15, wherein the patient is administered a dose in the range of 5 mg/kg to 100 mg/kg of the at least one E-selectin antagonist once a day for 7 days.
39. The method according to any one of Embodiments 10 to 15, wherein the patient is administered a dose in the range of 5 mg/kg to 100 mg/kg of the at least one E-selectin antagonist twice a day for 7 days.
40. The method according to any one of Embodiments 10 to 15, wherein the patient is administered a dose in the range of 5 mg/kg to 100 mg/kg of the at least one E-selectin antagonist for less than 7 days.
41. The method according to any one of Embodiments 10 to 15, wherein the patient is administered a dose in the range of 5 mg/kg to 100 mg/kg of the at least one E-selectin antagonist once a day for less than 7 days.
42. The method according to any one of Embodiments 10 to 15, wherein the patient is administered a dose in the range of 5 mg/kg to 100 mg/kg of the at least one E-selectin antagonist twice a day for less than 7 days.
43. The method according to any one of Embodiments 10 to 15, wherein the patient is administered a dose in the range of 5 mg/kg to 100 mg/kg of the at least one E-selectin antagonist for more than 7 days.
44. The method according to any one of Embodiments 10 to 15, wherein the patient is administered a dose in the range of 5 mg/kg to 100 mg/kg of the at least one E-selectin antagonist once a day for more than 7 days.
45. The method according to any one of Embodiments 10 to 15, wherein the patient is administered a dose in the range of 5 mg/kg to 100 mg/kg of the at least one E-selectin antagonist twice a day for more than 7 days.
46. The method according to any one of Embodiments 10 to 15, wherein the patient is administered a dose of 20 mg/kg of the at least one E-selectin antagonist twice a day.
47. The method according to any one of Embodiments 10 to 15, wherein the patient is administered a dose of 20 mg/kg of the at least one E-selectin antagonist twice a day for 7 days.
48. The method according to any one of Embodiments 1 to 8, wherein the patient is administered a dose of 20 mg/kg of the at least one E-selectin antagonist twice a day, wherein the at least one E-selectin antagonist is chosen from Compound A:

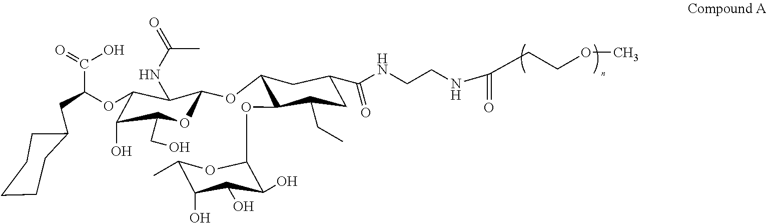

and pharmaceutically acceptable salts thereof.
49. The method according to any one of Embodiments 1 to 8, wherein the patient is administered a dose of 20 mg/kg of the at least one E-selectin antagonist twice a day for 7 days, wherein the at least one E-selectin antagonist is chosen from Compound A:

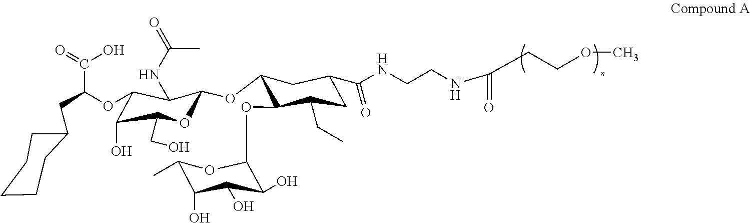

and pharmaceutically acceptable salts thereof.
50. The method according to any one of Embodiments 1 to 49, wherein the patient possesses one or more of the following characteristics: an age in the range of 18 to 75 years; documented COVID-19 pneumonia, defined as upper respiratory tract specimen (nasopharyngeal swab (NPS) or viral throat swab) positive for COVID-19 and imaging (CXR/CT scan) suggestive of COVID-19 pneumonia; confirmed coronavirus (SARS-CoV-2) infection; within 48 hours of beginning supplemental oxygen; currently hospitalized; requiring supplemental oxygen; having severe COVID-19 according to the World Health Organization (WHO) Interim Guidance with confirmation by real-time RT-PCR assay; respiratory distress; respiratory rate (RR)≥30 beats/min; oxygen saturation level less than 93% in resting state; partial pressure of oxygen (PaO₂)/oxygen concentration (FiO₂)≤300 mmHg.
51. The method according to any one of Embodiments 1 to 50, wherein the patient does not possess one or more of the following characteristics: in the opinion of at least two investigators, unlikely to survive for >48 hours from screening; severe chronic respiratory disease (e.g. COPD or other) requiring supplemental oxygen and/or having required mechanical ventilation pre-COVID-19 infection; currently on invasive mechanical ventilation; hypotension defined as systolic blood pressure <90 mmHg on two sequential readings at least 4 hours apart; total Bilirubin ≤3×upper limit of normal (ULN), Creatinine Clearance ≥30 mL/min/1.73 m²; pregnant or breastfeeding; known diagnosis of an acute thrombosis on admission, concurrent dual antithrombotic therapy (aspirin or P2Y12 inhibitor plus anticoagulation to treat deep venous thrombosis or pulmonary embolism (single antiplatelet or anticoagulant agent at prophylactic dose is permitted); concomitant use of thrombolytic therapy; concomitant therapeutic systemic anticoagulant therapy (e.g. heparin, warfarin, direct thrombin inhibitors and direct factor Xa inhibitors); history of recent major bleeding, defined in accordance with the criteria of the International Society on Thrombosis and Hemostasis (ISTH); history of bleeding disorder thought to impose excessive bleeding risk, as per investigator discretion; hemodynamic instability, defined as inability to maintain mean arterial pressure.

In some embodiments, a method of treating COVID-19 patients with antagonists of E-selectin is disclosed, the method comprising administering to a COVID-19 patient an effective amount of at least one E-selectin antagonist and/or a pharmaceutical composition comprising at least one E-selectin antagonist.

In some embodiments, a method of treating a disease, disorder, and/or condition associated with COVID-19 with antagonists of E-selectin is disclosed, the method comprising administering to a COVID-19 patient an effective amount of at least one E-selectin antagonist and/or a pharmaceutical composition comprising at least one E-selectin antagonist.

In some embodiments, the disease, disorder, and/or condition associated with COVID-19 is acute lung injury. In some embodiments, the disease, disorder, and/or condition associated with COVID-19 is acute respiratory distress syndrome. In some embodiments, the disease, disorder, and/or condition associated with COVID-19 is pneumonia. In some embodiments, the disease, disorder, and/or condition associated with COVID-19 is ARDS pneumonia.

In some embodiments, a method of treating acute respiratory distress syndrome with antagonists of E-selectin is disclosed, the method comprising administering to an acute respiratory distress syndrome patient an effective amount of at least one E-selectin antagonist and/or a pharmaceutical composition comprising at least one E-selectin antagonist.

In some embodiments, a method of treating acute lung injury with antagonists of E-selectin is disclosed, the method comprising administering to an acute lung injury patient an effective amount of at least one E-selectin antagonist and/or a pharmaceutical composition comprising at least one E-selectin antagonist.

In some embodiments, a method of treating pneumonia with antagonists of E-selectin is disclosed, the method comprising administering to a pneumonia patient an effective amount of at least one E-selectin antagonist and/or a pharmaceutical composition comprising at least one E-selectin antagonist. In some embodiments, the pneumonia is ARDS pneumonia.

In some embodiments, the patient is intravenously administered the at least one E-selectin antagonist. In some embodiments, the patient is intravenously administered the at least one E-selectin antagonist once a day. In some embodiments, the patient is intravenously administered the at least one E-selectin antagonist twice a day.

In some embodiments, the patient possesses one or more (e.g., one, two, three, four, five, six, seven, eight, or nine) of the following characteristics: an age in the range of 18 to 75 years; documented COVID-19 pneumonia, defined as upper respiratory tract specimen (nasopharyngeal swab (NPS) or viral throat swab) positive for COVID-19 and imaging (CXR/CT scan) suggestive of COVID-19 pneumonia; confirmed coronavirus (SARS-CoV-2) infection; within 48 hours of beginning supplemental oxygen; currently hospitalized; requiring supplemental oxygen; having severe COVID-19 according to the World Health Organization (WHO) Interim Guidance with confirmation by real-time RT-PCR assay; respiratory distress; respiratory rate (RR)≥30 beats/min; oxygen saturation level less than 93% in resting state; partial pressure of oxygen (PaO₂)/oxygen concentration (FiO₂)≤300 mmHg.

In some embodiments, the patient does not possess one or more (e.g., one, two, three, four, five, six, seven, eight, nine, ten, or eleven) of the following characteristics: in the opinion of at least two investigators, unlikely to survive for >48 hours from screening; severe chronic respiratory disease (e.g. COPD or other) requiring supplemental oxygen and/or having required mechanical ventilation pre-COVID-19 infection; currently on invasive mechanical ventilation; hypotension defined as systolic blood pressure <90 mmHg on two sequential readings at least 4 hours apart, total Bilirubin ≤3× upper limit of normal (ULN), Creatinine Clearance ≥30 mL/min/1.73 m²; pregnant or breastfeeding; known diagnosis of an acute thrombosis on admission; concurrent dual antithrombotic therapy (aspirin or P2Y12 inhibitor plus anticoagulation to treat deep venous thrombosis or pulmonary embolism (single antiplatelet or anticoagulant agent at prophylactic dose is permitted); concomitant use of thrombolytic therapy; concomitant therapeutic systemic anticoagulant therapy (e.g. heparin, warfarin, direct thrombin inhibitors and direct factor Xa inhibitors); history of recent major bleeding, defined in accordance with the criteria of the International Society on Thrombosis and Hemostasis (ISTH); history of bleeding disorder thought to impose excessive bleeding risk, as per investigator discretion; hemodynamic instability, defined as inability to maintain mean arterial pressure.

In some embodiments, the patient possesses one or more (e.g., one, two, three, four, five, six, seven, eight, or nine) of the following characteristics: an age in the range of 18 to 75 years; documented COVID-19 pneumonia, defined as upper respiratory tract specimen (nasopharyngeal swab (NPS) or viral throat swab) positive for COVID-19 and imaging (CXR/CT scan) suggestive of COVID-19 pneumonia; confirmed coronavirus (SARS-CoV-2) infection; within 48 hours of beginning supplemental oxygen; currently hospitalized; requiring supplemental oxygen; having severe COVID-19 according to the World Health Organization (WHO) Interim Guidance with confirmation by real-time RT-PCR assay; respiratory distress; respiratory rate (RR) ≥30 beats/min; oxygen saturation level less than 93% in resting state; partial pressure of oxygen (PaO₂)/oxygen concentration (FiO₂)≤300 mmHg; and the patient does not possess one or more (e.g., one, two, three, four, five, six, seven, eight, nine, ten, or eleven) of the following characteristics: in the opinion of at least two investigators, unlikely to survive for >48 hours from screening; severe chronic respiratory disease (e.g. COPD or other) requiring supplemental oxygen and/or having required mechanical ventilation pre-COVID-19 infection; currently on invasive mechanical ventilation; hypotension defined as systolic blood pressure <90 mmHg on two sequential readings at least 4 hours apart; total Bilirubin ≤3× upper limit of normal (ULN), Creatinine Clearance ≥30 mL/min/1.73 m²; pregnant or breastfeeding; known diagnosis of an acute thrombosis on admission; concurrent dual antithrombotic therapy (aspirin or P2Y12 inhibitor plus anticoagulation to treat deep venous thrombosis or pulmonary embolism (single antiplatelet or anticoagulant agent at prophylactic dose is permitted); concomitant use of thrombolytic therapy; concomitant therapeutic systemic anticoagulant therapy (e.g. heparin, warfarin, direct thrombin inhibitors and direct factor Xa inhibitors); history of recent major bleeding, defined in accordance with the criteria of the International Society on Thrombosis and Hemostasis (ISTH); history of bleeding disorder thought to impose excessive bleeding risk, as per investigator discretion; hemodynamic instability, defined as inability to maintain mean arterial pressure.

In some embodiments, the administration begins within 48 hours of administration of supplemental oxygen to the patient.

In some embodiments, the patient is administered a dose in the range of 5 mg/kg to 100 mg/kg (e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin antagonist. In some embodiments, the patient is administered a dose in the range of 5 mg/kg to 100 mg/kg (e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin antagonist for less than 7 days. In some embodiments, the patient is administered a dose in the range of 5 mg/kg to 100 mg/kg (e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin antagonist for 7 days. In some embodiments, the patient is administered a dose in the range of 5 mg/kg to 100 mg/kg (e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin antagonist for more than 7 days.

In some embodiments, the patient is administered a dose in the range of 5 mg/kg to 100 mg/kg (e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin antagonist once a day. In some embodiments, the patient is administered a dose in the range of 5 mg/kg to 100 mg/kg (e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin antagonist once a day for less than 7 days. In some embodiments, the patient is administered a dose in the range of 5 mg/kg to 100 mg/kg (e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin antagonist once a day for 7 days. In some embodiments, the patient is administered a dose in the range of 5 mg/kg to 100 mg/kg (e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin antagonist once a day for more than 7 days.

In some embodiments, the patient is administered a dose in the range of 5 mg/kg to 100 mg/kg (e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin antagonist twice a day. In some embodiments, the patient is administered a dose in the range of 5 mg/kg to 100 mg/kg (e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin antagonist twice a day for less than 7 days. In some embodiments, the patient is administered a dose in the range of 5 mg/kg to 100 mg/kg (e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin antagonist twice a day for 7 days. In some embodiments, the patient is administered a dose in the range of 5 mg/kg to 100 mg/kg (e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin antagonist twice a day for more than 7 days.

In some embodiments, the patient is administered a dose of 20 mg/kg of the at least one E-selectin antagonist twice a day. In some embodiments, the patient is administered a dose of 20 mg/kg of the at least one E-selectin antagonist twice a day for less than 7 days. In some embodiments, the patient is administered a dose of 20 mg/kg of the at least one E-selectin antagonist twice a day for 7 days. In some embodiments, the patient is administered a dose of 20 mg/kg of the at least one E-selectin antagonist twice a day for more than 7 days.

In some embodiments, the patient is administered a dose of 20 mg/kg of the at least one E-selectin antagonist twice a day, wherein the at least one E-selectin antagonist is chosen from Compound A:

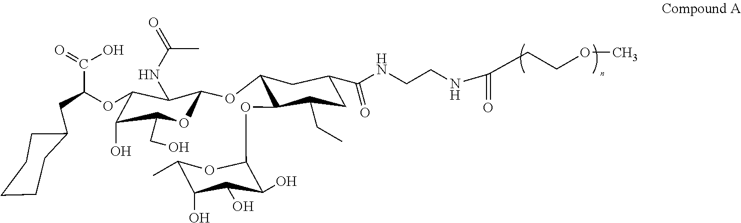

and pharmaceutically acceptable salts thereof.

In some embodiments, the patient is administered a dose of 20 mg/kg of the at least one E-selectin antagonist twice a day for 7 days, wherein the at least one E-selectin antagonist is chosen from Compound A:

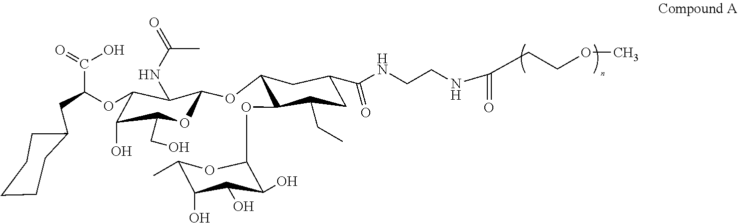

and pharmaceutically acceptable salts thereof.

The E-selectin ligand is a carbohydrate structure that contains the epitope shared by sialyl Le^{a} and sialyl Le^{x}. Carbohydrates are secondary gene products synthesized by enzymes known as glycosyltransferases which are the primary gene products coded for by DNA. Each glycosyltransferase adds a specific monosaccharide in a specific stereochemical linkage to a specific donor carbohydrate chain.

Non-limiting examples of suitable E-selectin antagonists include small molecules, such as nucleic acids, peptides, polypeptides, peptidomimetics, carbohydrates, glycomimetics, lipids and other organic (carbon containing) or inorganic molecules. Suitably, the E-selectin antagonist is selected from antigen-binding molecules that are immuno-interactive with a selectin, peptides that bind to the selectin and that block cell-cell adhesion, and carbohydrate or peptide mimetics of selectin ligands. In some embodiments, the E-selectin antagonist reduces the expression of a selectin gene or the level or functional activity of an expression product of that gene. For example, the E-selectin antagonist may antagonize the function of the selectin, including reducing or abrogating the activity of at least one of its ligand-binding sites.

In some embodiments, the E-selectin antagonist inhibits an activity of E-selectin or inhibits the binding of E-selectin to one or more E-selectin ligands (which in turn may inhibit a biological activity of E-selectin).

In some embodiments, the E-selectin antagonist is chosen from compounds of Formula (I):

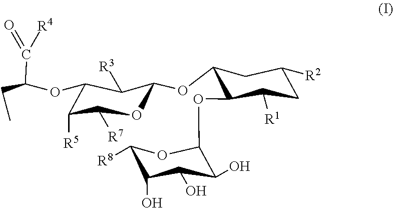

isomers of Formula (I), tautomers of Formula (I), and pharmaceutically acceptable salts of any of the foregoing, wherein:
R¹ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R² is chosen from H, -M, and -L-M;
R³ is chosen from —OH, —NH₂, —OC(═O)Y¹, —NHC(═O)Y¹, and —NHC(═O)NHY¹ groups, wherein Y¹ is chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups;
R⁴ is chosen from —OH and —NZ¹Z² groups, wherein Z¹ and Z², which may be identical or different, are each independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Z¹ and Z² may together form a ring;
R⁵ is chosen from C₃₋₈ cycloalkyl groups;
R⁶ is chosen from —OH, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R⁷ is chosen from —CH₂OH, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R⁸ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
L is chosen from linker groups; and
M is a non-glycomimetic moiety chosen from polyethylene glycol, thiazolyl, chromenyl, —C(═O)NH(CH₂)₁₋₄NH₂, C₁₋₈ alkyl, and —C(═O)OY groups, wherein Y is chosen from C₁₋₄ alkyl, C₂₋₄ alkenyl, and C₂₋₄ alkynyl groups.

In some embodiments, the E-selectin antagonist is chosen from compounds of Formula (I), wherein the non-glycomimetic moiety comprises polyethylene glycol.

In some embodiments, the E-selectin antagonist is chosen from compounds of Formula (I), wherein L is —C(═O)NH(CH₂)₁₋₄NHC(═O)— and the non-glycomimetic moiety comprises polyethylene glycol.

In some embodiments, the E-selectin antagonist is chosen from compounds of Formula (Ia):

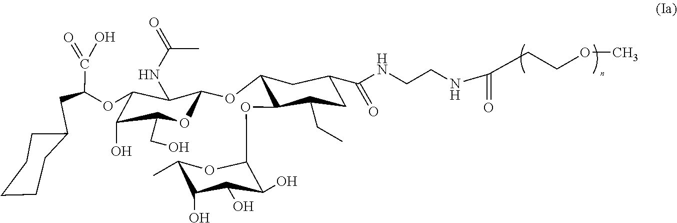

and pharmaceutically acceptable salts thereof, wherein n is chosen from integers ranging from 1 to 100. In some embodiments, n is chosen from 4, 8, 12, 16, 20, 24, and 28. In some embodiments n is 12.

In some embodiments, the E-selectin antagonist is chosen from Compound A.

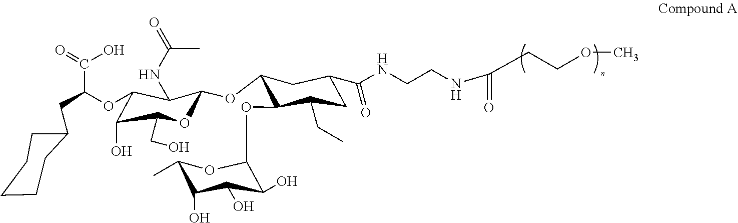

and pharmaceutically acceptable salts thereof.

In some embodiments, the E-selectin antagonist is a heterobifunctional antagonist chosen from compounds of Formula (II):

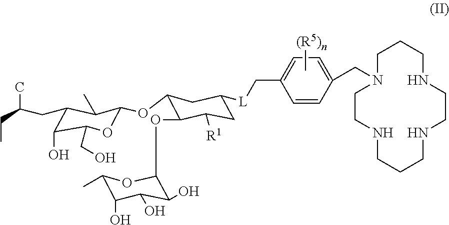

isomers of Formula (II), tautomers of Formula (II), and pharmaceutically acceptable salts of any of the foregoing, wherein:
R¹ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R² is chosen from —OH, —NH₂, —OC(═O)Y¹, —NHC(═O)Y¹, and —NHC(═O)NHY¹ groups, wherein Y¹ is chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups;
R³ is chosen from —CN, —CH₂CN, and —C(═O)Y² groups, wherein Y² is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Z¹ and Z² may together form a ring;
R⁴ is chosen from C₃₋₈ cycloalkyl groups;
R⁵ is independently chosen from H, halo, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
n is chosen from integers ranging from 1 to 4; and
L is chosen from linker groups.

In some embodiments, the E-selectin antagonist is a heterobifunctional antagonist chosen from compounds of Formula (IIa):

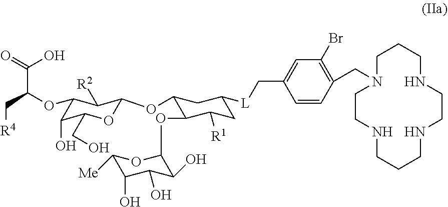

and pharmaceutically acceptable salts thereof.

In some embodiments, the E-selectin antagonist is chosen from Compound B:

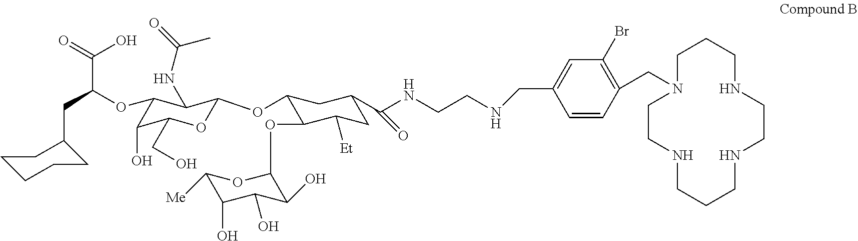 [SMILES: CCC1CC(C(=O)NCCNCc2ccc(CN3CCCNCCNCCCNCC3)c(Br)c2)CC(OC2OC(CO)C(O)C(O[C@@H](CC3CCCCC3)C(=O)O)C2NC(C)=O)C1OC1OC(C)C(O)C(O)C1O]

and pharmaceutically acceptable salts thereof.

In some embodiments, the E-selectin antagonist is a heterobifunctional pan-selectin antagonist chosen from compounds of Formula (III):

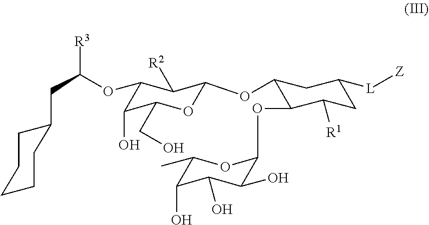

isomers of Formula (III), tautomers of Formula (III), and pharmaceutically acceptable salts of any of the foregoing, wherein:
R¹ is chosen from H, C₁₋₈ alkyl, C₂₋₈ alkenyl, C₂₋₈ alkynyl, C₄₋₁₆cycloalkylalkyl

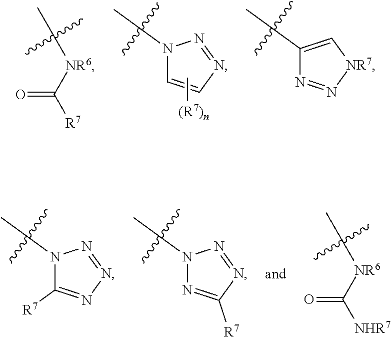

groups;
R² is chosen from C₁₋₈ alkyl, C₂₋₈ alkenyl, C₂₋₈ alkynyl, C₄₋₁₆cycloalkylalkyl, —OH, —OX¹, halo, —NH₂, —OC(═O)X¹, —NHC(═O)X¹, and —NHC(═O)NHX¹ groups, wherein X¹ is chosen from C₁₋₈ alkyl, C₂₋₈ alkenyl, C₂₋₈ alkynyl, C₄₋₁₆cycloalkylalkyl, C₂₋₁₂ heterocyclyl, C₆₋₁₈ aryl, and C₁₋₁₃ heteroaryl groups;
R³ is chosen from —CN, —CH₂CN, and —C(═O)X² groups, wherein X² is chosen from C₁₋₈ alkyl, C₂₋₈ alkenyl, C₂₋₈ alkynyl, —OY², —NHOH, —NHOCH₃, —NHCN, and —NY²Y³ groups, wherein Y² and Y³, which may be identical or different, are independently chosen from H, C₁₋₈ alkyl, C₂₋₈ alkenyl, C₂₋₈ alkynyl, and C₄₋₁₆ cycloalkylalkyl groups, wherein Y² and Y³ may join together to form a ring;
R⁶ is chosen from H, C₁₋₈ alkyl, C₂₋₈ alkenyl, C₂₋₈ alkynyl, C₄₋₁₆cycloalkylalkyl, and —C(═O)R⁷ groups;
each R⁷ is independently chosen from H, C₁₋₈ alkyl, C₂₋₈ alkenyl, C₂₋₈ alkynyl, C₄₋₁₆ cycloalkylalkyl,

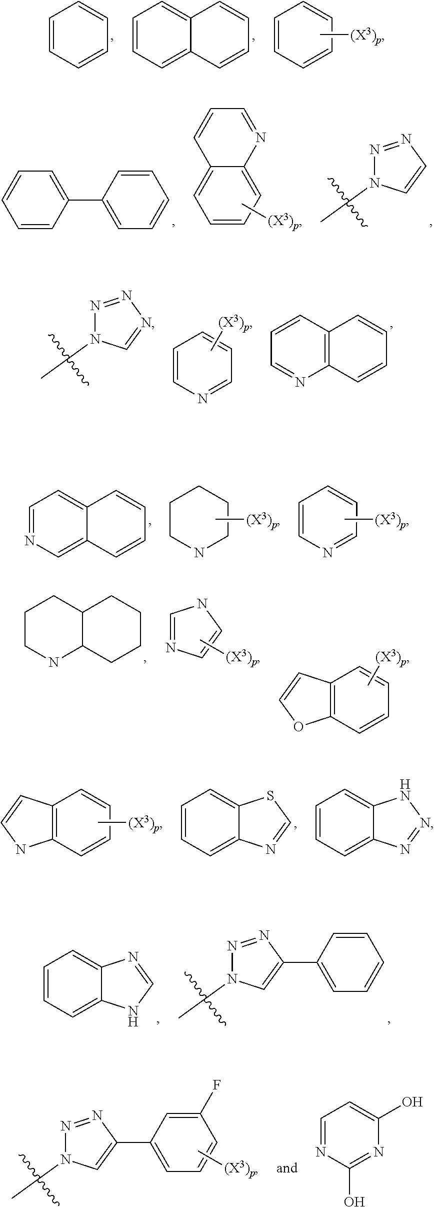

groups, wherein each X³ is independently chosen from H, —OH, Cl, F, N₃, —NH₂, C₁₋₈ alkyl, C₂₋₈ alkenyl, C₂₋₈ alkynyl, C₆₋₁₄ aryl, —OC₁₋₈ alkyl, —OC₂₋₈ alkenyl, —OC₂₋₈ alkynyl, and —OC₆₋₁₄ aryl groups, wherein any of the above ring compounds may be substituted with one to three groups independently chosen from Cl, F, C₁₋₈ alkyl, C₂₋₈ alkenyl, C₂₋₈ alkynyl, C₆₋₁₄ aryl, and —OY⁴ groups, wherein Y⁴ is chosen from H, C₁₋₈ alkyl, C₂₋₈ alkenyl, C₂₋₈ alkynyl, and C₆₋₁₄ aryl groups;
n is chosen from integers ranging from 0 to 2;
p is chosen from integers ranging from 0 to 3;
L is chosen from linker groups; and
Z is chosen from benzyl amino sulfonic acid groups.

Benzyl amino sulfonic acids (BASAs) are low molecular weight sulfated compounds which have the ability to interact with a selectin. The interaction modulates or assists in the modulation (e.g., inhibition or enhancement) of a selectin-mediated function (e.g., an intercellular interaction). They exist as either their protonated acid form, or as a sodium salt, although sodium may be replaced with potassium or any other pharmaceutically acceptable counterion.

Further disclosure regarding BASAs suitable for the disclosed compounds may be found in U.S. Reissue Patent No. RE44,778, issued Feb. 25, 2014, and U.S. Publication No. US2018/0369205, published Dec. 27, 2018, which are hereby incorporated by reference.

In some embodiments, the E-selectin antagonist is a heterobifunctional pan-selectin antagonist chosen from compounds of Formula (IIIa):

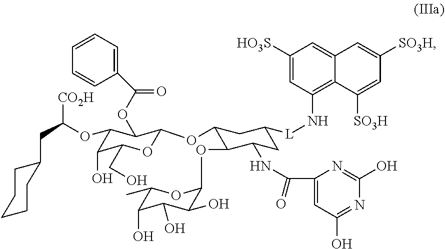

tautomers of Formula (IIIa), and pharmaceutically acceptable salts of any of the foregoing.

In some embodiments, the E-selectin antagonist is a heterobifunctional pan-selectin antagonist chosen from Compound C:

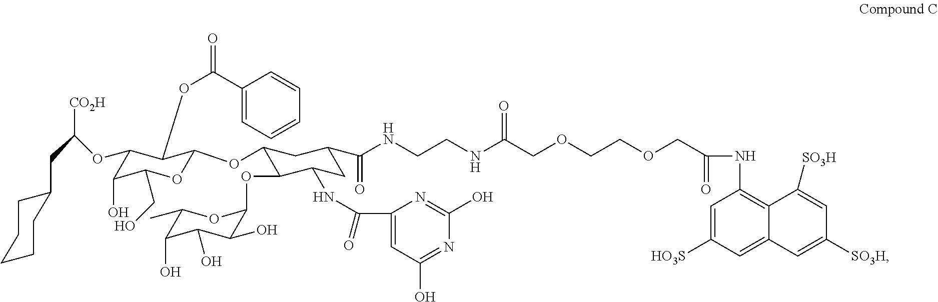 [SMILES: CC1OC(OC2C(NC(=O)c3cc(O)nc(O)n3)CC(C(=O)NCCNC(=O)COCCOCC(=O)Nc3cc(S(=O)(=O)O)cc4cc(S(=O)(=O)O)cc(S(=O)(=O)O)c34)CC2OC2OC(CO)C(O)C(O[C@@H](CC3CCCCC3)C(=O)O)C2OC(=O)c2ccccc2)C(O)C(O)C1O]

tautomers of Compound C, and pharmaceutically acceptable salts of any of the foregoing.

In some embodiments, the linker groups of Formula (I), Formula (II), and/or Formula (III) are independently chosen from groups comprising spacer groups, such spacer groups as, for example, —(CH₂)ₚ— and —O(CH₂)ₚ—, wherein p is chosen from integers ranging from 1 to 30. In some embodiments, p is chosen from integers ranging from 1 to 20.

Other non-limiting examples of spacer groups include carbonyl groups and carbonyl-containing groups such as, for example, amide groups.

In some embodiments, the linker group of Formula (I), Formula (II), and/or Formula (III) is chosen from

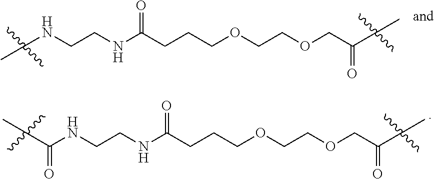 [SMILES: *NCCNC(=O)CCCOCCOCC(*)=O ; *C(=O)COCCOCCCC(=O)NCCNC(*)=O]

In some embodiments, the linker group of Formula (I), Formula (II), and/or Formula (III) is chosen from

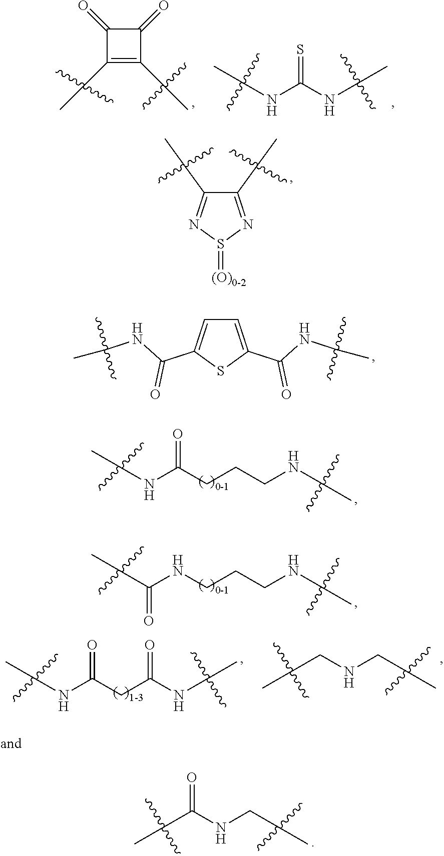

Other linker groups, such as, for example, polyethylene glycols (PEGs) and —C(═O)—NH—(CH₂)ₚ—C(═O)—NH—, wherein p is chosen from integers ranging from 1 to 30, or wherein p is chosen from integers ranging from 1 to 20, will be familiar to those of ordinary skill in the art and/or those in possession of the present disclosure.

In some embodiments, the linker group of Formula (I), Formula (II), and/or Formula (III) is chosen from

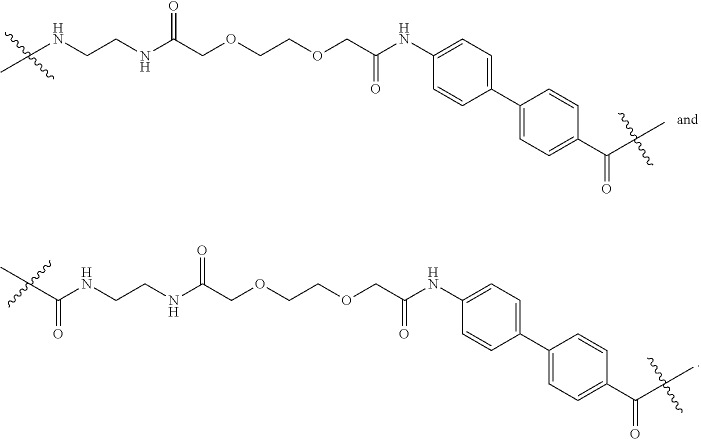 [SMILES: *NCCNC(=O)COCCOCC(=O)Nc1ccc(-c2ccc(C(*)=O)cc2)cc1 ; *C(=O)NCCNC(=O)COCCOCC(=O)Nc1ccc(-c2ccc(C(*)=O)cc2)cc1]

In some embodiments, the linker group of Formula (I), Formula (II), and/or Formula (III) is chosen from

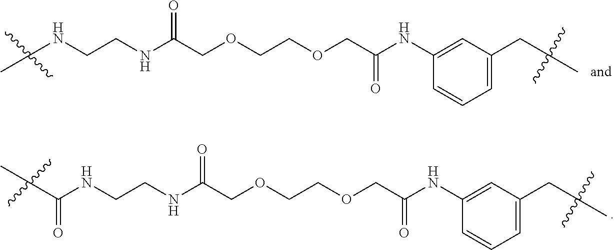 [SMILES: *Cc1cccc(NC(=O)COCCOCC(=O)NCCN*)c1 ; *Cc1cccc(NC(=O)COCCOCC(=O)NCCNC(*)=O)c1]

In some embodiments, the linker group of Formula (I), Formula (II), and/or Formula (III) is chosen from

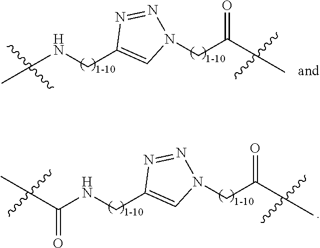 [SMILES: *NCc1cn(CC(*)=O)nn1 ; *C(=O)Cn1cc(CNC(*)=O)nn1]

In some embodiments, the linker group of Formula (I), Formula (II), and/or Formula (III) is chosen from —C(═O)NH(CH₂)₂NH—, —CH₂NHCH₂—, and —C(═O)NHCH₂—. In some embodiments, the linker group is —C(═O)NH(CH₂)₂NH—.

In some embodiments, the E-selectin antagonist is chosen from compounds of Formula (IV):

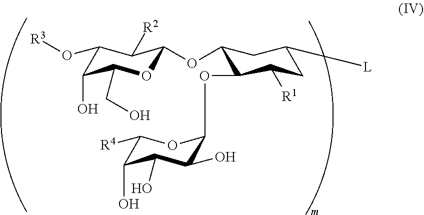

prodrugs of Formula (IV), isomers of Formula (IV), tautomers of Formula (IV), and pharmaceutically acceptable salts of any of the foregoing, wherein
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NY¹Y² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;
each R³, which may be identical or different, is independently chosen from

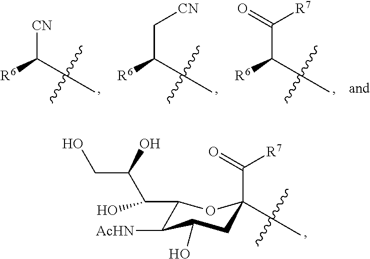

wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is chosen from integers ranging from 2 to 256; and
L is chosen from linker groups.

In some embodiments, the E-selectin antagonist is chosen from compounds of Formula (V):

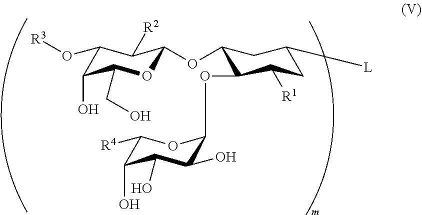

prodrugs of Formula (V), isomers of Formula (V), tautomers of Formula (V), and pharmaceutically acceptable salts of any of the foregoing, wherein
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NY¹Y² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;
each R³, which may be identical or different, is independently chosen from

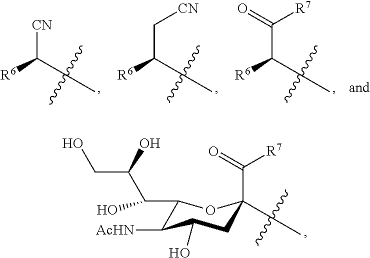

wherein each R⁶ which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is 2; and
L is chosen from

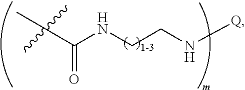

wherein Q is a chosen from

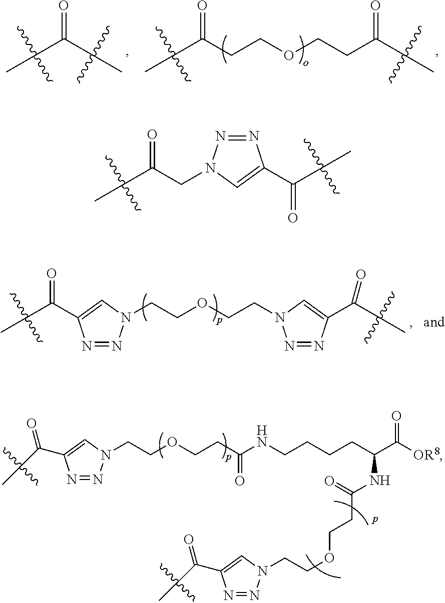

wherein R⁸ is chosen from H, C₁₋₈ alkyl, C₆₋₁₈ aryl, C₇₋₁₉ arylalkyl, and C₁₋₁₃ heteroaryl groups and each p, which may be identical or different, is independently chosen from integers ranging from 0 to 250.

In some embodiments, the E-selectin antagonist of Formula (IV) or Formula (V) is chosen from compounds of the following Formula (IVa/Va) (see definitions of L and m for Formula (IV) or (V) above):

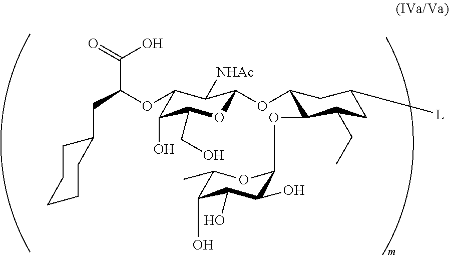

In some embodiments, the E-selectin antagonist of Formula (IV) or Formula (V) is chosen from compounds of the following Formula (IVb/Vb) (see definitions of L and m for Formula (IV) or (V) above):

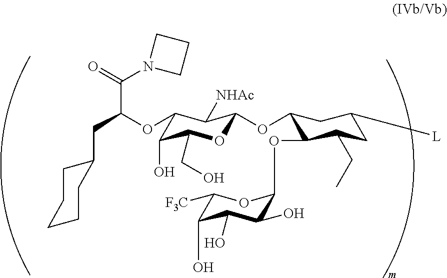

In some embodiments, the E-selectin antagonist is Compound D:

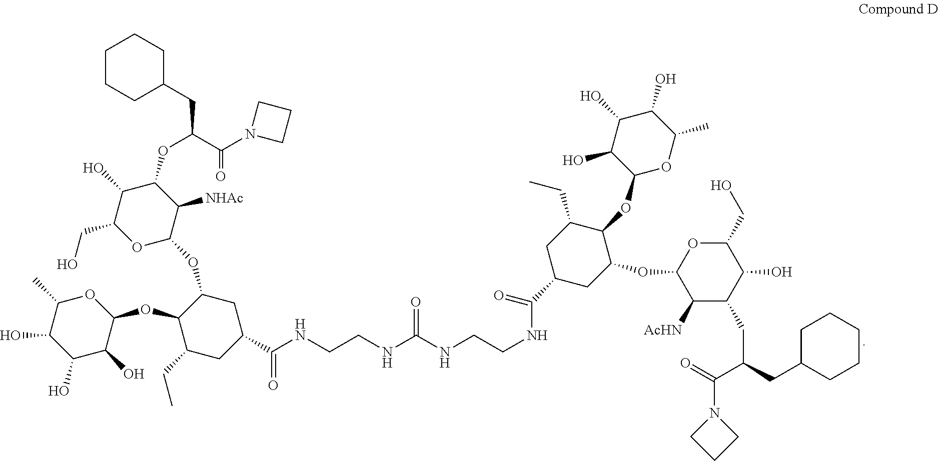 [SMILES: CC[C@H]1C[C@@H](C(=O)NCCNC(=O)NCCNC(=O)[C@@H]2C[C@H](CC)[C@@H](O[C@@H]3O[C@@H](C)[C@@H](O)[C@@H](O)[C@@H]3O)[C@H](O[C@@H]3O[C@H](CO)[C@H](O)[C@H](O[C@@H](CC4CCCCC4)C(=O)N4CCC4)[C@H]3NC(C)=O)C2)C[C@@H](O[C@@H]2O[C@H](CO)[C@H](O)[C@H](C[C@@H](CC3CCCCC3)C(=O)N3CCC3)[C@H]2NC(C)=O)[C@@H]1O[C@@H]1O[C@@H](C)[C@@H](O)[C@@H](O)[C@@H]1O]

In some embodiments, the E-selectin antagonist is a heterobifunctional inhibitor of E-selectin and Galectin-3, chosen from compounds of Formula (VI):

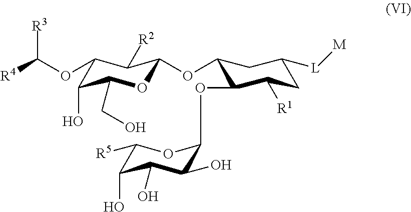

prodrugs of Formula (VI), isomers of Formula (VI), tautomers of Formula (VI), and pharmaceutically acceptable salts of any of the foregoing, wherein
R¹ is chosen from HK C₁₋₈ alkyl, C₂₋₈ alkenyl, C₂₋₈ alkynyl, C₁₋₈ haloalkyl, C₂₋₈ haloalkenyl, C₂₋₈ haloalkynyl,

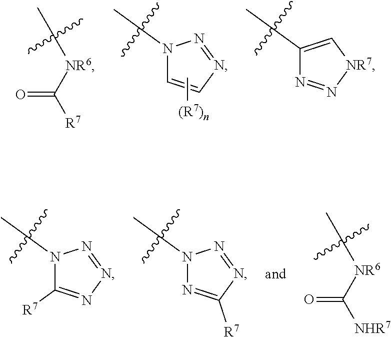

groups, wherein n is chosen from integers ranging from 0 to 2, R⁶ is chosen from H, C₁₋₈ alkyl, C₂₋₈ alkenyl, C₂₋₈ alkynyl, C₄₋₁₆ cycloalkylalkyl, and —C(═O)R⁷ groups, and each R⁷ is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
R² is chosen from —OH, —OY¹, halo, —NH₂, —NY¹Y², —OC(═O)Y¹, —NHC(═O)Y¹, and —NHC(═O)NHY¹ groups, wherein Y¹ and Y², which may be the same or different, are independently chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups, wherein Y¹ and Y² may join together along with the nitrogen atom to which they are attached to form a ring;
R³ is chosen from —CN, —CH₂CN, and —C(═O)Y³ groups, wherein Y³ is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₇₋₁₂ arylalkyl groups, wherein Z¹ and Z² may join together along with the nitrogen atom to which they are attached to form a ring;
R⁴ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, and C₆₋₁₈ aryl groups;
R⁵ is chosen from —CN, C₁₋₈ alkyl, and C₁₋₄ haloalkyl groups;
M is chosen from

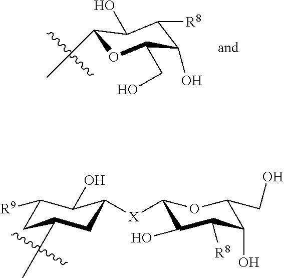

groups, wherein X is chosen from O and S, and R⁸ and R⁹, which may be identical or different, are independently chosen from C₆₋₁₈ aryl, C₁₋₁₃ heteroaryl, C₇₋₁₉ arylalkyl, C₇₋₁₉ arylalkoxy, C₂₋₁₄ heteroarylalkyl, C₂₋₁₄ heteroarylalkoxy, and —NHC(═O)Y⁴ groups, wherein Y⁴ is chosen from C₁₋₈ alkyl, C₂₋₁₂ heterocyclyl, C₆₋₁₈ aryl, and C₁₋₁₃ heteroaryl groups; and
L is chosen from linker groups.

In some embodiments, the E-selectin antagonist is chosen from compounds having the following Formulae:

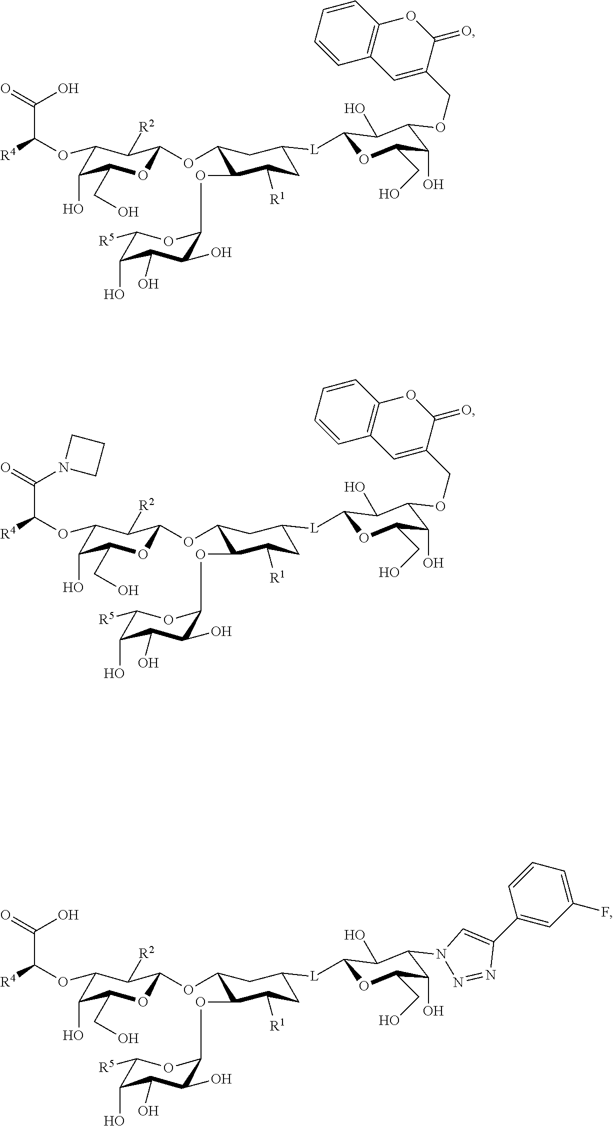

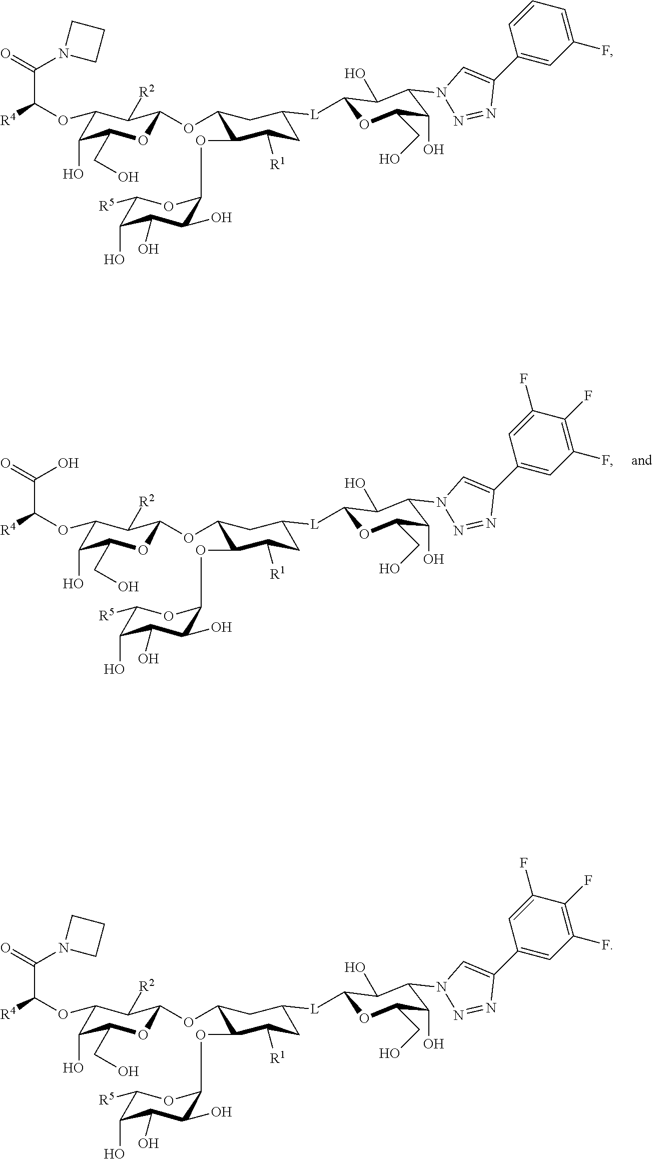

In some embodiments, the E-selectin antagonist is chosen from compounds having the following Formulae:

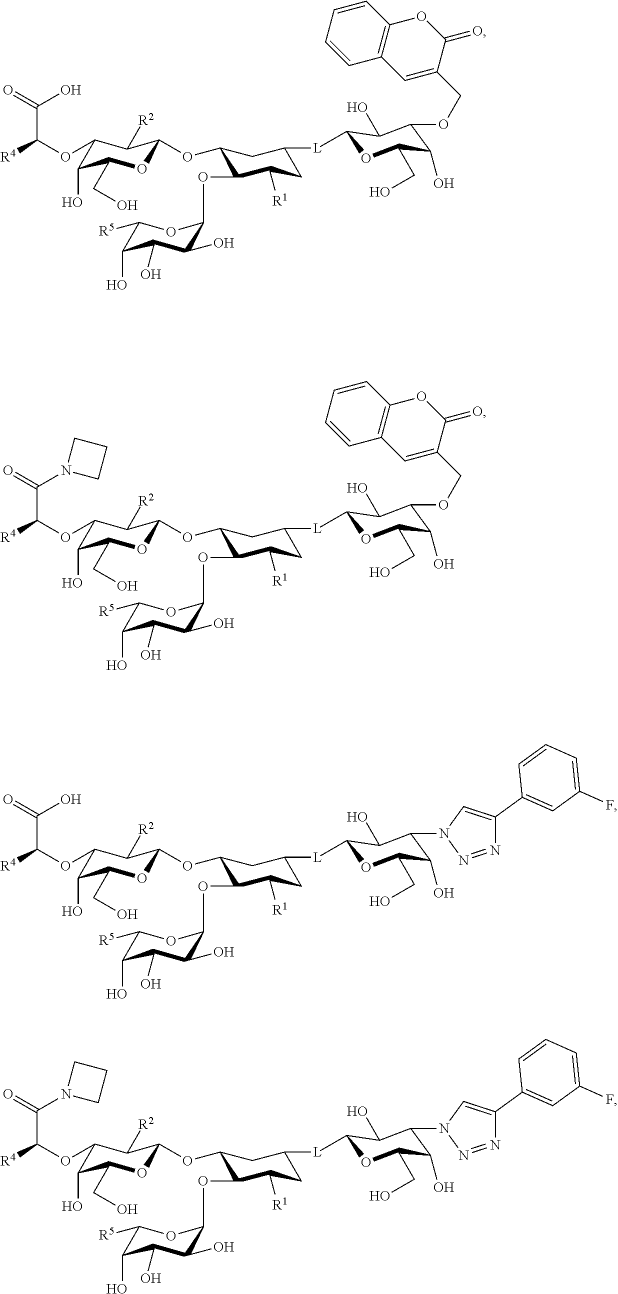

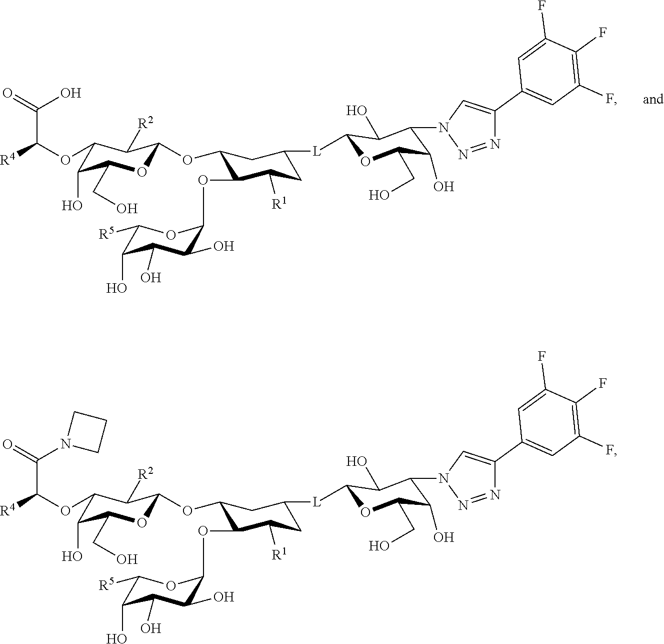

and pharmaceutically acceptable salts of any of the foregoing.

In some embodiments, the E-selectin antagonist is chosen from compounds having the following Formulae:

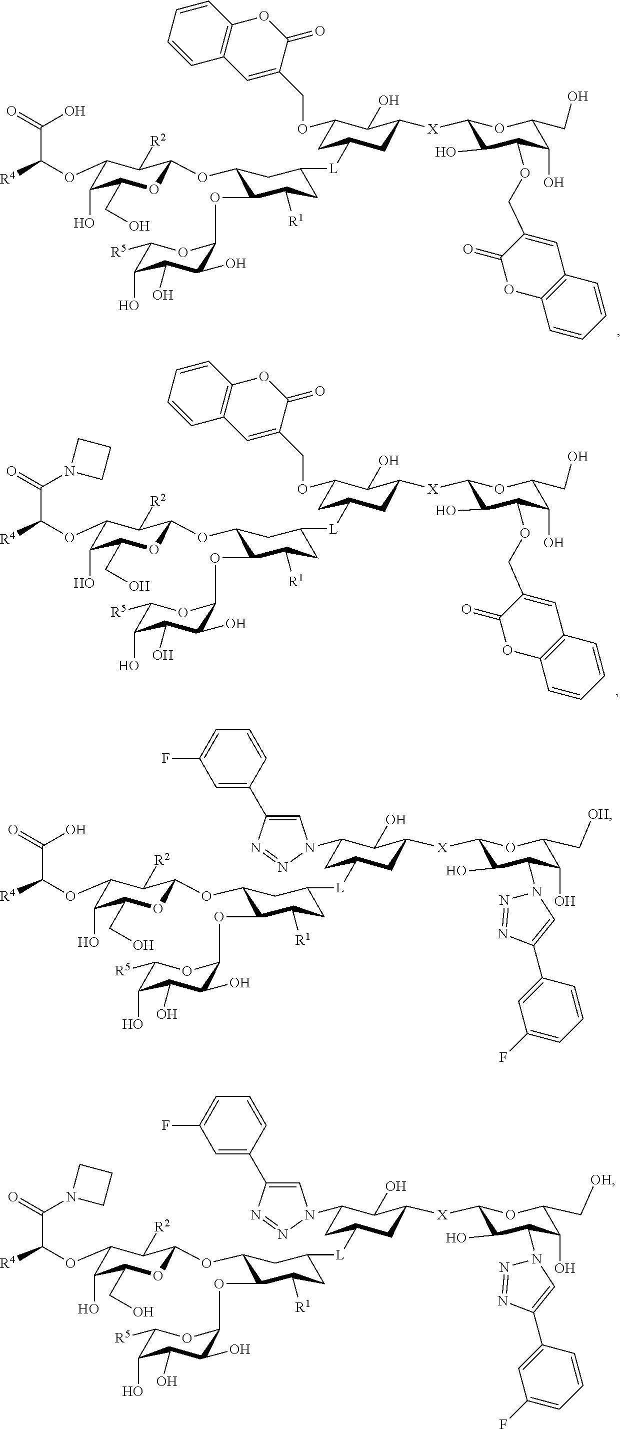

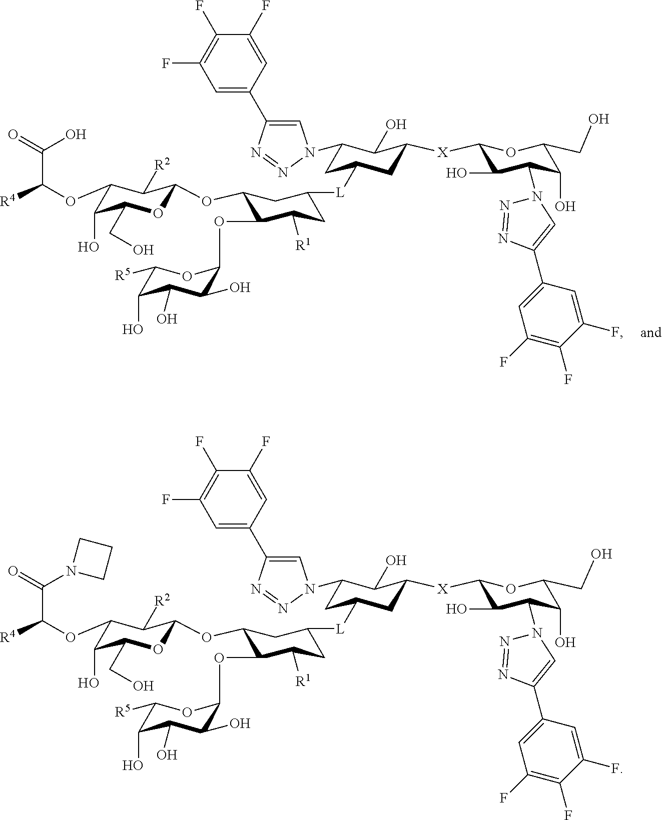

In some embodiments, the E-selectin antagonist is chosen from compounds having the following Formulae:

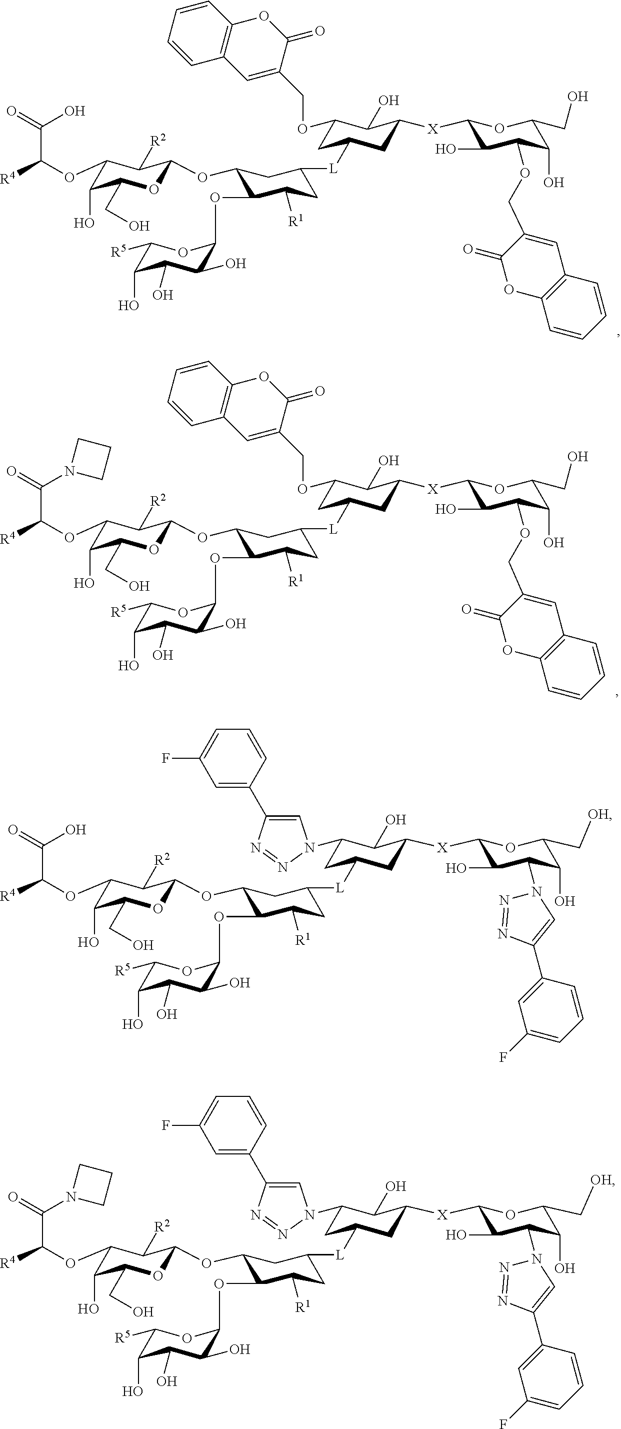

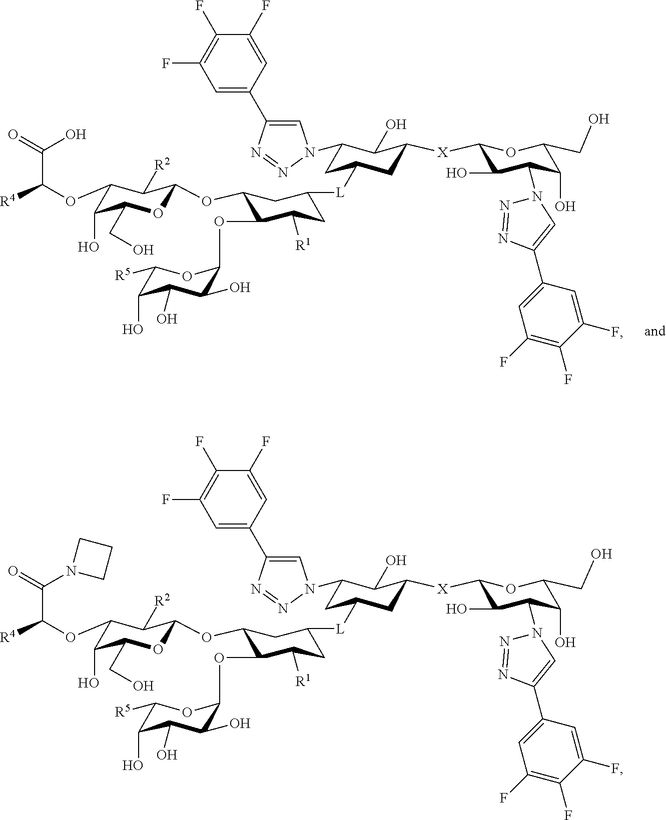

and pharmaceutically acceptable salts of any of the foregoing.

In some embodiments, the E-selectin antagonist is Compound E:

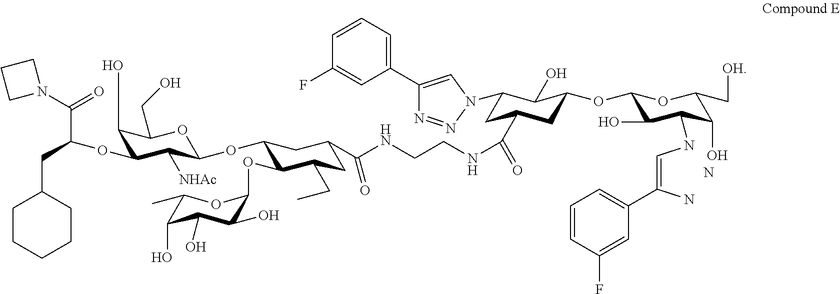

In some embodiments, the E-selectin antagonist is chosen from compounds of Formula (VII):

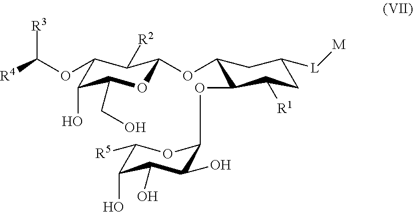

prodrugs of Formula (VII), isomers of Formula (VII), tautomers of Formula (VII), and pharmaceutically acceptable salts of any of the foregoing, wherein
R¹ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl,

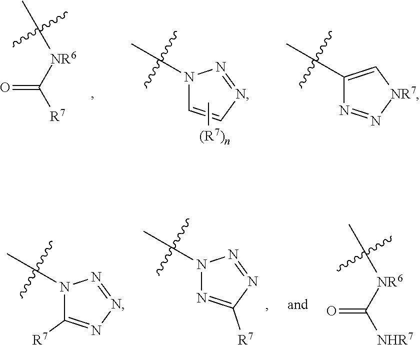

groups, wherein n is chosen from integers ranging from 0 to 2, R⁶ is chosen from H, C₁₋₈ alkyl, C₂₋₈ alkenyl, C₂₋₈ alkynyl, C₄₋₁₆ cycloalkylalkyl, and —C(═O)R⁷ groups, and each R⁷ is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
R² is chosen from —OH, —OY¹, halo, —NH₂, —NY¹Y², —OC(═O)Y¹, —NHC(═O)Y¹, and —NHC(═O)NHY¹ groups, wherein Y¹ and Y², which may be the same or different, are independently chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups, or Y¹ and Y² join together along with the nitrogen atom to which they are attached to form a ring;
R³ is chosen from —CN, —CH₂CN, and —C(═O)Y³ groups, wherein Y³ is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₇₋₁₂ arylalkyl groups, or Z¹ and Z² join together along with the nitrogen atom to which they are attached to form a ring;
R⁴ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, and C₆₋₁₈ aryl groups;
R⁵ is chosen from —CN, C₁₋₈ alkyl, and C₁₋₄ haloalkyl groups;
M is chosen from

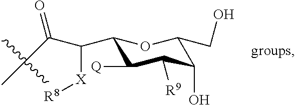

wherein
X is chosen from —O—, —S—, —C—, and —N(R¹⁰)—, wherein R¹⁰ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups,
Q is chosen from H, halo, and —OZ³ groups, wherein Z³ is chosen from H and C₁₋₈ alkyl groups,
R⁸ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₇₋₁₉ arylalkyl, and C₂₋₁₄ heteroarylalkyl groups, wherein the C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₇₋₁₉ arylalkyl, and C₂₋₁₄ heteroarylalkyl groups are optionally substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, —C(═O)NZ⁴Z⁵, and —SO₂Z⁴ groups, wherein Z⁴ and Z, which may be identical or different, are independently chosen from H, C₁₋₈ alkyl, and C₁₋₈ haloalkyl groups, or Z⁴ and Z⁵ join together along with the nitrogen atom to which they are attached to form a ring,
R⁹ is chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from R¹¹, C₁₋₈ alkyl, C₁₋₈ haloalkyl, —C(═O)OZ⁶, and —C(═O)NZ⁶Z⁷ groups, wherein R¹¹ is independently chosen from C₆₋₁₈ aryl groups optionally substituted with one or more groups independently chosen from halo, C₁₋₈ alkyl, —OZ⁸, —C(═O)OZ⁸, and —C(═O)NZ⁸Z⁹ groups, wherein Z⁶, Z⁷, Z⁸ and Z⁹, which may be identical or different, are independently chosen from H and C₁₋₈. alkyl groups, or Z⁶ and Z⁷ join together along with the nitrogen atom to which they are attached to form a ring and/or Z⁸ and Z⁹ join together along with the nitrogen atom to which they are attached to form a ring, and
wherein each of Z³, Z⁴, Z⁵, Z⁶, Z⁷, Z⁸, and Z⁹ is optionally substituted with one or more groups independently chosen from halo and —OR¹² groups, wherein R¹² is independently chosen from H and C₁₋₈ alkyl groups; and
L is chosen from linker groups.

In some embodiments of Formula (VII), M is chosen from

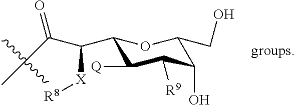

In some embodiments of Formula (VII), M is chosen from

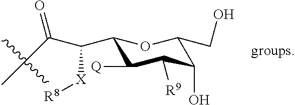

In some embodiments of Formula (VII), linker groups may be chosen from groups comprising spacer groups, such spacer groups as, for example, —(CH₂)ₜ— and —O(CH₂)ₜ—, wherein t is chosen from integers ranging from 1 to 20. Other non-limiting examples of spacer groups include carbonyl groups and carbonyl-containing groups such as, for example, amide groups. A non-limiting example of a spacer group is

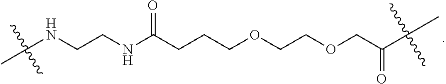 [SMILES: *NCCNC(=O)CCCOCCOCC(*)=O]

In some embodiments of Formula (VII), the linker group is chosen from

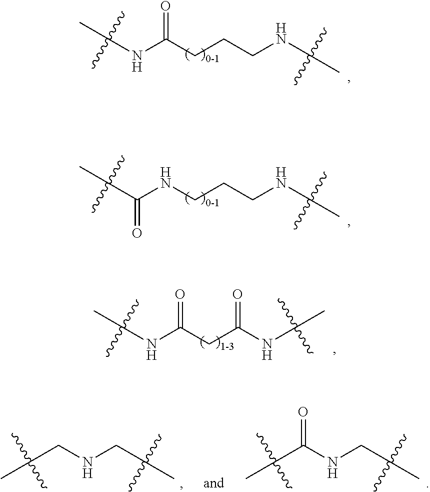

In some embodiments of Formula (VII), the linker group is chosen from polyethylene glycols (PEGs), —C(═O)NH(CH₂)ᵥO—, —C(═O)NH(CH₂)ᵥNHC(═O), —C(═O)NHC(═O)(CH₂)NH—, and —C(═O)NH(CH₂)ᵥ—C(═O)NH-groups, wherein v is chosen from integers ranging from 2 to 20. In some embodiments, v is chosen from integers ranging from 2 to 4. In some embodiments, v is 2. In some embodiments, v is 3. In some embodiments, v is 4.

In some embodiments of Formula (VII), the linker group is

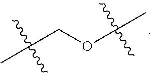 [SMILES: *CO*]

In some embodiments of Formula (VII), the linker group is

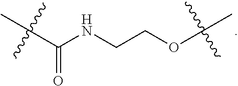 [SMILES: *OCCNC(*)=O]

In some embodiments of Formula (VII), the linker group is

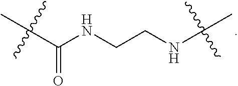 [SMILES: *NCCNC(*)=O]

In some embodiments of Formula (VII), the linker group is

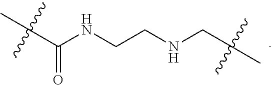 [SMILES: *CNCCNC(*)=O]

In some embodiments of Formula (VII), the linker group is

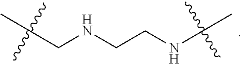 [SMILES: *CNCCN*]

In some embodiments of Formula (VII), the linker group is

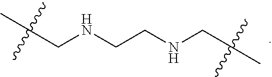 [SMILES: *CNCCNC*]

In some embodiments of Formula (VII), the linker group is

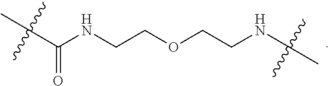 [SMILES: *NCCOCCNC(*)=O]

In some embodiments of Formula (VII), the linker group is

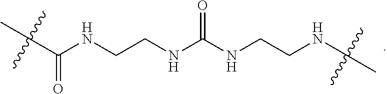 [SMILES: *NCCNC(=O)NCCNC(*)=O]

In some embodiments of Formula (VII), the linker group is

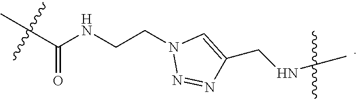 [SMILES: *NCc1cn(CCNC(*)=O)nn1]

Figures and examples illustrating the synthesis of compounds of Formula (VII) are shown in PCT International Application Publication No. WO 2020/139962, which is incorporated by reference herein.

In some embodiments, the E-selectin antagonist is a multimeric inhibitor of E-selectin, Galectin-3, and/or CXCR4, chosen from compounds of Formula (VIII):

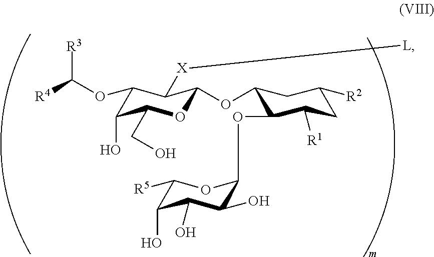

prodrugs of Formula (VIII), and pharmaceutically acceptable salts of any of the foregoing, wherein
each R¹, which may be identical or different, is independently chosen from H, C₁₋₁₂ alkyl, C₂₋₁₂ alkenyl, C₂₋₁₂ alkynyl, C₁₋₈ haloalkyl, C₂₋₈ haloalkenyl, C₂₋₈ haloalkynyl,

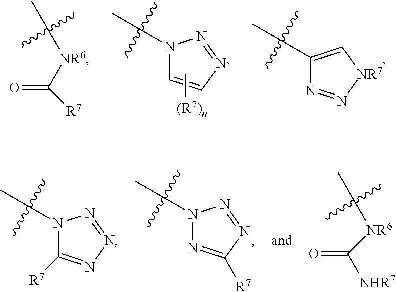

groups, wherein each n, which may be identical or different, is chosen from integers ranging from 0 to 2, each R⁶, which may be identical or different, is independently chosen from H, C₁₋₈ alkyl, C₂₋₈ alkenyl, C₂₋₈ alkynyl, C₄₋₁₆ cycloalkylalkyl, and —C(═O)R⁷ groups, and each R⁷, which may be identical or different, is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
each R², which may be identical or different, is independently chosen from H, a non-glycomimetic moiety, and a linker-non-glycomimetic moiety, wherein each non-glycomimetic moiety, which may be identical or different, is independently chosen from galectin-3 inhibitors, CXCR4 chemokine receptor inhibitors, polyethylene glycol, thiazolyl, chromenyl, C₁₋₈ alkyl, R⁸, C₆₋₁₈ aryl-R⁸, C₁₋₁₂ heteroaryl-R⁸,

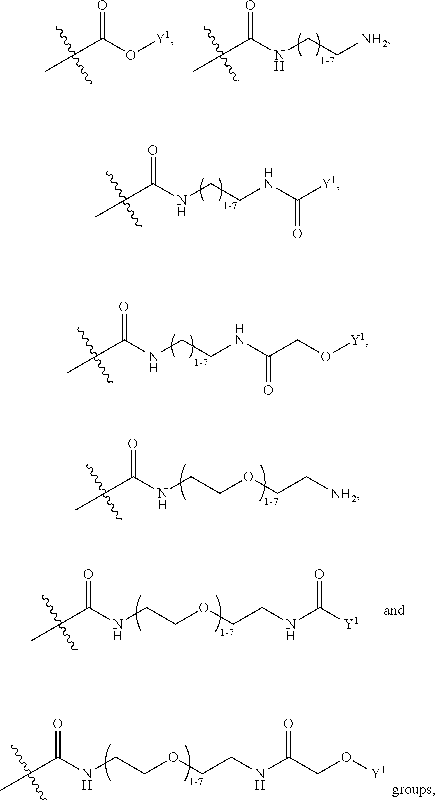

wherein each Y¹, which may be identical or different, is independently chosen from C₁₋₄ alkyl, C₂₋₄ alkenyl, and C₂₋₄ alkynyl groups and wherein each R⁸, which may be identical or different, is independently chosen from C₁₋₁₂ alkyl groups substituted with at least one substituent chosen from —OH, —OSO₃Q, —OPO₃Q₂, —CO₂Q, and —SO₃Q groups and C₂₋₁₂ alkenyl groups substituted with at least one substituent chosen from —OH, —OSO₃Q, —OPO₃Q₂, —CO₂Q, and —SO₃Q groups, wherein each Q, which may be identical or different, is independently chosen from H and pharmaceutically acceptable cations;
each R³, which may be identical or different, is independently chosen from —CN, —CH₂CN, and —C(═O)Y² groups, wherein each Y², which may be identical or different, is independently chosen from C₁₋₈ alkyl, C₂₋₈ alkenyl, C₂₋₈ alkynyl, —OZ¹, —NHOH, —NHOCH₃, —NHCN, and —NZ¹Z² groups, wherein each Z¹ and Z², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, and C₇₋₁₂ arylalkyl groups, wherein Z¹ and Z² may join together along with the nitrogen atom to which they are attached to form a ring;
each R⁴, which may be identical or different, is independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₄₋₁₆ cycloalkylalkyl, and C₆₋₁₈ aryl groups;
each R⁵, which may be identical or different, is independently chosen from —CN, C₁₋₁₂ alkyl, and C₁₋₁₂ haloalkyl groups;
each X, which may be identical or different, is independently chosen from —O— and —N(R⁹)—, wherein each R⁹, which may be identical or different, is independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
m is chosen from integers ranging from 2 to 256; and
L is independently chosen from linker groups.

In some embodiments of Formula (VIII), at least one linker groups is chosen from groups comprising spacer groups, such spacer groups as, for example, —(CH₂)z- and —O(CH₂)z—, wherein z is chosen from integers ranging from 1 to 250. Other non-limiting examples of spacer groups include carbonyl groups and carbonyl-containing groups such as, for example, amide groups. A non-limiting example of a spacer group is

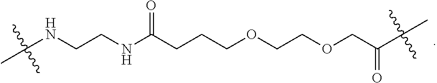 [SMILES: *NCCNC(=O)CCCOCCOCC(*)=O]

In some embodiments of Formula (VIII), at least one linker group is chosen from

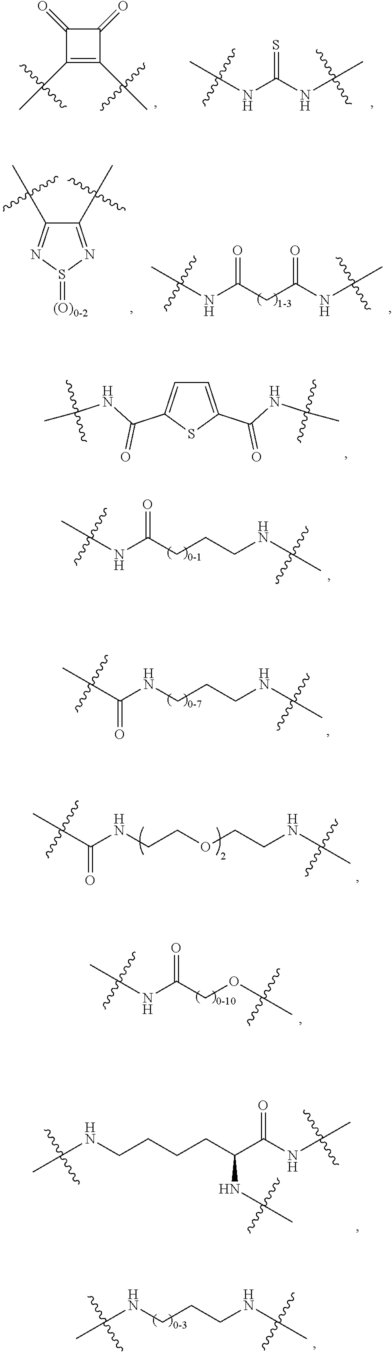

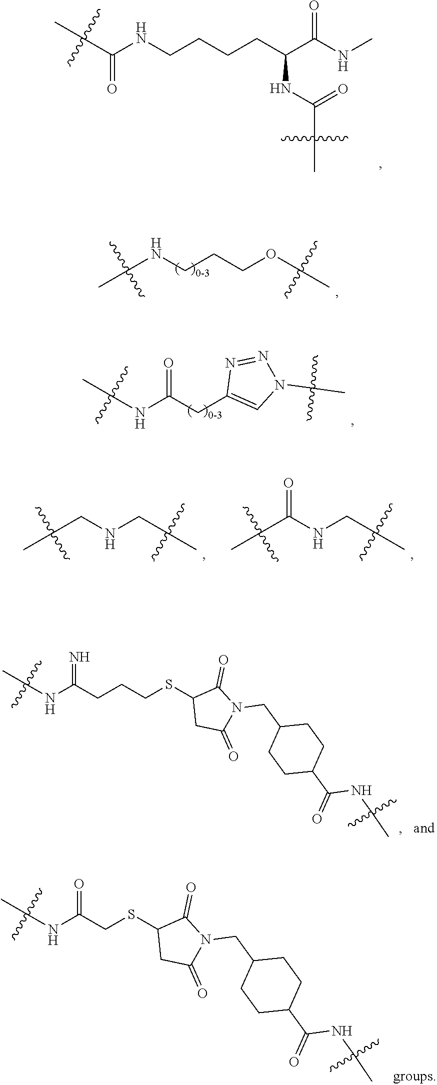

Other linker groups for certain embodiments of Formula (VIII), such as, for example, polyethylene glycols (PEGs) and —C(═O)—NH—(CH₂)_{z}—C(═O)—NH—, wherein z is chosen from integers ranging from 1 to 250, will be familiar to those of ordinary skill in the art and/or those in possession of the present disclosure.

In some embodiments of Formula (VIII), at least one linker group is

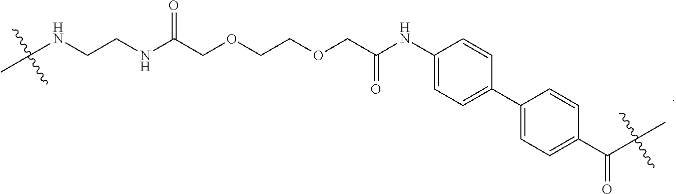 [SMILES: *NCCNC(=O)COCCOCC(=O)Nc1ccc(-c2ccc(C(*)=O)cc2)cc1]

In some embodiments of Formula (VIII), at least one linker group is

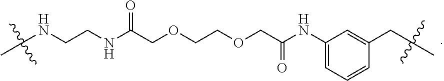 [SMILES: *Cc1cccc(NC(=O)COCCOCC(=O)NCCN*)c1]

In some embodiments of Formula (VIII), at least one linker group is chosen from —C(═O)NH(CH₂)₂NH—, —CH₂NHCH₂—, and —C(═O)NHCH₂—. In some embodiments of Formula (VIII), at least one linker group is —C(═O)NH(CH₂)₂NH—.

In some embodiments of Formula (VIII), L is chosen from dendrimers. In some embodiments of Formula (VIII), L is chosen from polyamidoamine (“PAMAM”) dendrimers. In some embodiments of Formula (VIII), L is chosen from PAMAM dendrimers comprising succinamic. In some embodiments of Formula (VIII), L is PAMAM GO generating a tetramer. In some embodiments of Formula (VIII), L is PAMAM G1 generating an octamer. In some embodiments of Formula (VIII), L is PAMAM G2 generating a 16-mer. In some embodiments of Formula (VIII), L is PAMAM G3 generating a 32-mer. In some embodiments of Formula (VIII), L is PAMAM G4 generating a 64-mer. In some embodiments, L is PAMAM G5 generating a 128-mer.

In some embodiments of Formula (VIII), m is 2 and L is chosen from

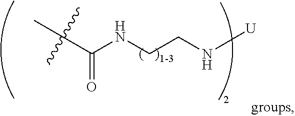

wherein U is chosen from

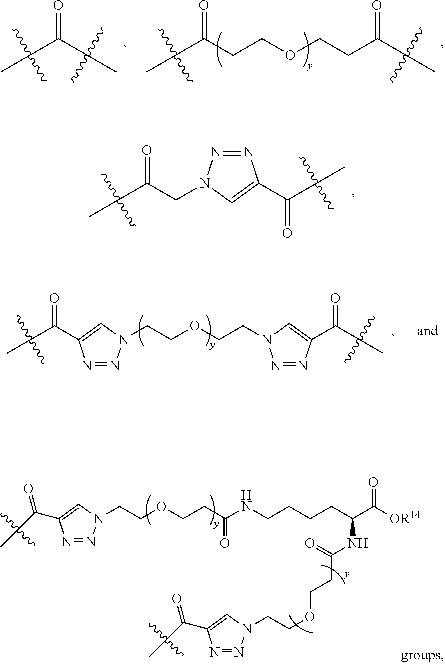

wherein R¹⁴ is chosen from H, C₁₋₈ alkyl, C₆₋₁₈ aryl, C₇₋₁₉ arylalkyl, and C₁₋₁₃ heteroaryl groups and each y, which may be identical or different, is independently chosen from integers ranging from 0 to 250. In some embodiments of Formula (VIII), R¹⁴ is chosen from C₁₋₈ alkyl. In some embodiments of Formula (VIII), R¹⁴ is chosen from C₇₋₁₉ arylalkyl. In some embodiments of Formula (VIII), R¹⁴ is H. In some embodiments of Formula (VIII), R¹⁴ is benzyl.

In some embodiments of Formula (VIII), L is chosen from

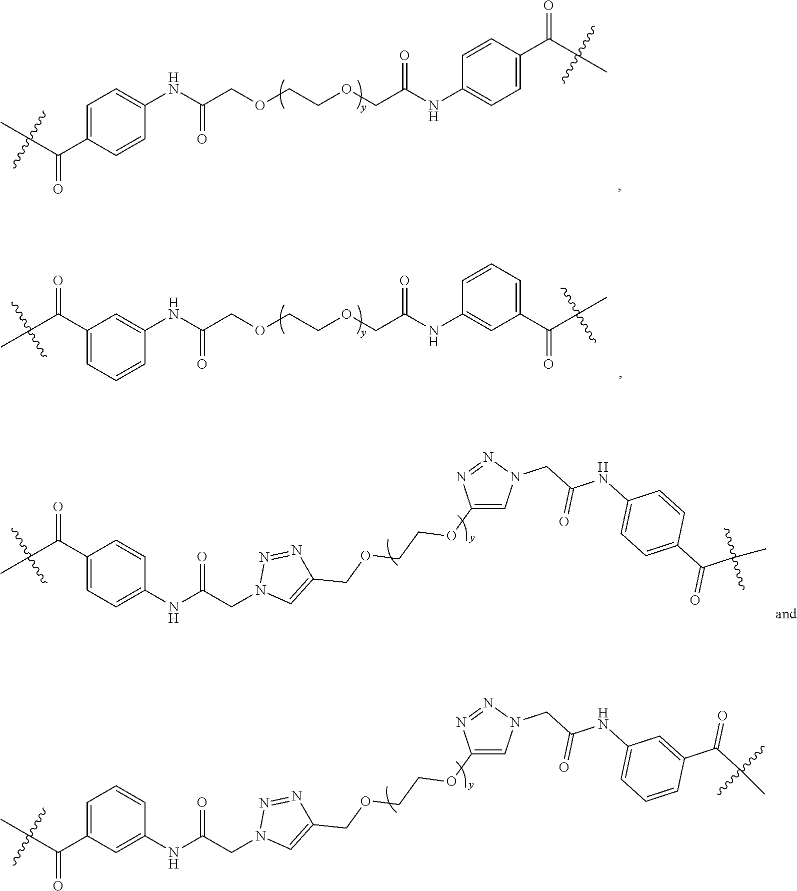

wherein y is chosen from integers ranging from 0 to 250.

In some embodiments of Formula (VIII), L is chosen from

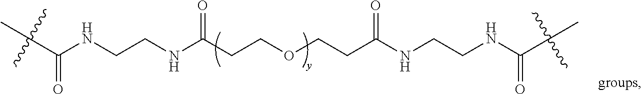

wherein y is chosen from integers ranging from 0 to 250.

In some embodiments of Formula (VIII), L is

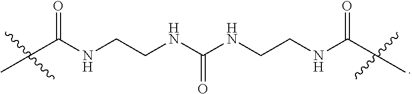 [SMILES: *C(=O)NCCNC(=O)NCCNC(*)=O]

In some embodiments of Formula (VIII), L is chosen from

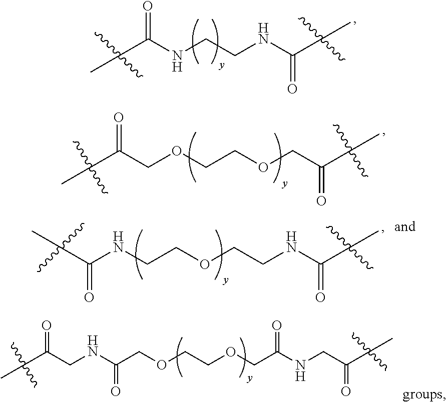

wherein y is chosen from integers ranging from 0 to 250.

In same embodiments of Formula (VIII), L is chosen from

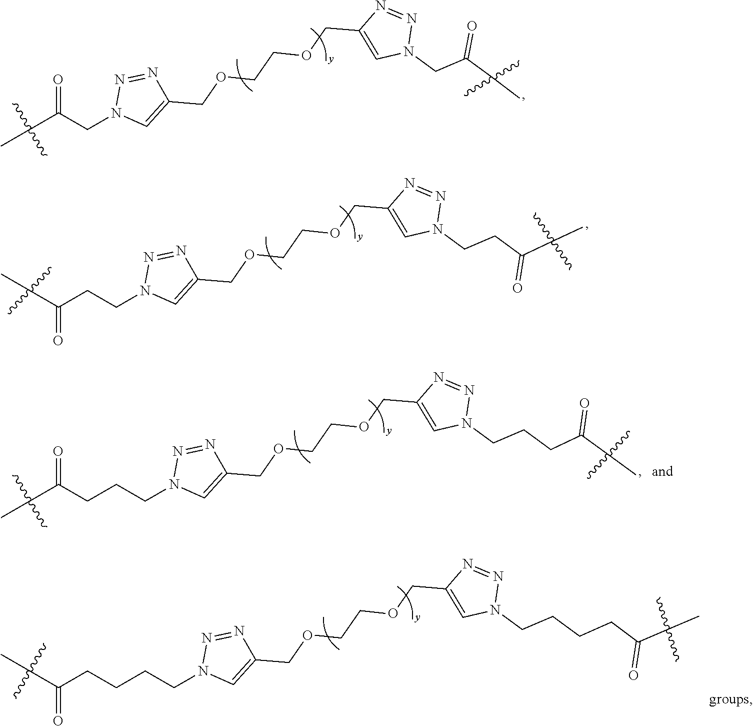

wherein y is chosen from integers ranging from 0 to 250.

In some embodiments of Formula (VIII), L is chosen from

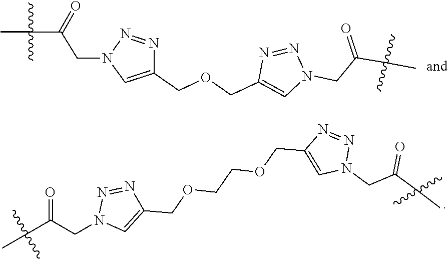 [SMILES: *C(=O)Cn1cc(COCc2cn(CC(*)=O)nn2)nn1 ; *C(=O)Cn1cc(COCCOCc2cn(CC(*)=O)nn2)nn1]

In some embodiments of Formula (VIII), L is

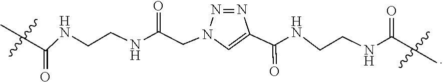 [SMILES: *C(=O)NCCNC(=O)Cn1cc(C(=O)NCCNC(*)=O)nn1]

In some embodiments of Formula (VIII), L is chosen from

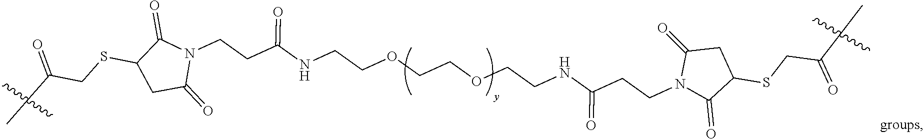

wherein y is chosen from integers ranging from 0 to 250.

In some embodiments of Formula (VIII), L is

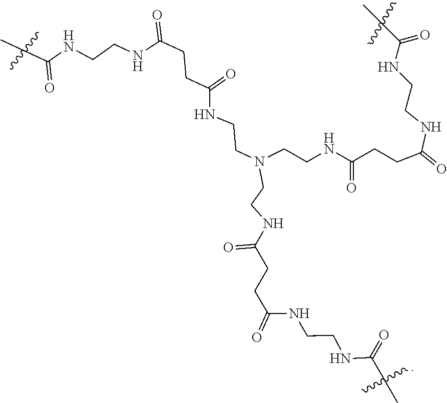 [SMILES: *C(=O)NCCNC(=O)CCC(=O)NCCN(CCNC(=O)CCC(=O)NCCNC(*)=O)CCNC(=O)CCC(=O)NCCNC(*)=O]

In some embodiments of Formula (VIII), L is

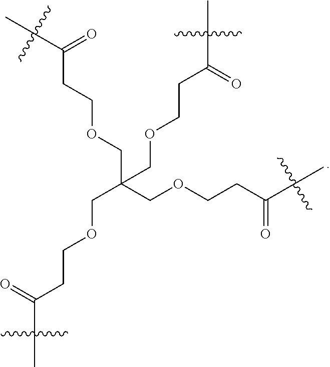 [SMILES: *C(=O)CCOCC(COCCC(*)=O)(COCCC(*)=O)COCCC(*)=O]

In same embodiments of Formula (VIII), L is

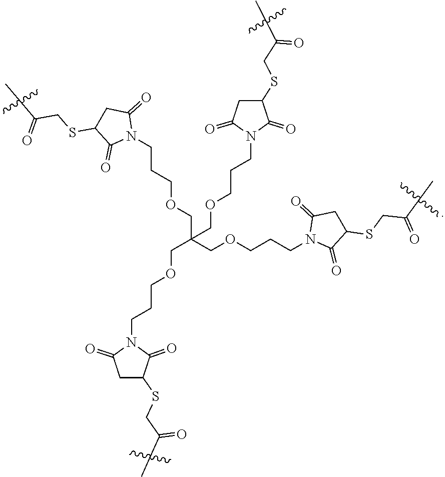 [SMILES: *C(=O)CSC1CC(=O)N(CCCOCC(COCCCN2C(=O)CC(SCC(*)=O)C2=O)(COCCCN2C(=O)CC(SCC(*)=O)C2=O)COCCCN2C(=O)CC(SCC(*)=O)C2=O)C1=O]

In some embodiments of Formula (VIII), L is chosen from

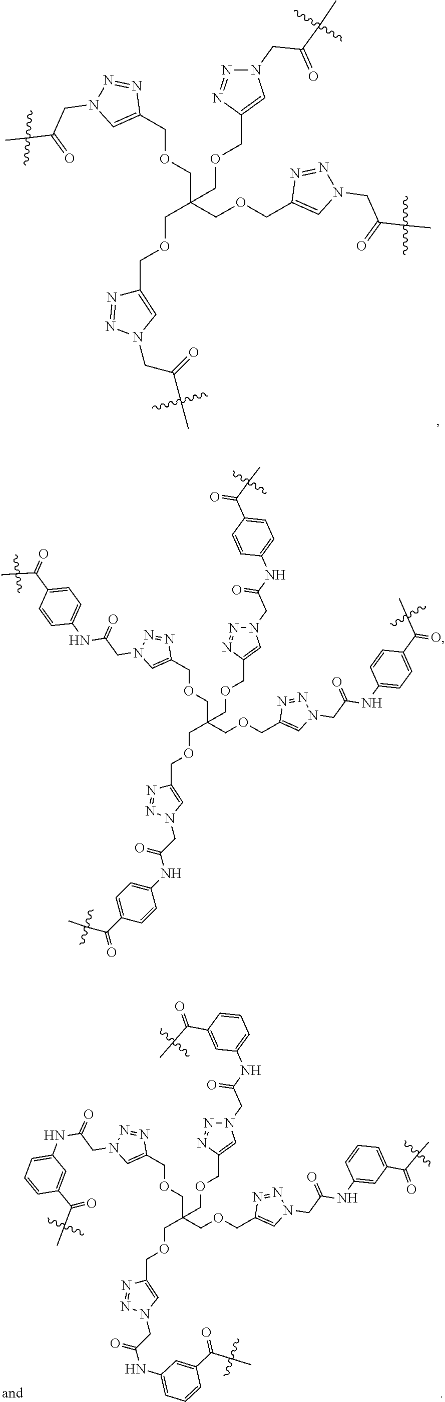 [SMILES: *C(=O)Cn1cc(COCC(COCc2cn(CC(*)=O)nn2)(COCc2cn(CC(*)=O)nn2)COCc2cn(CC(*)=O)nn2)nn1 ; *C(=O)c1ccc(NC(=O)Cn2cc(COCC(COCc3cn(CC(=O)Nc4ccc(C(*)=O)cc4)nn3)(COCc3cn(CC(=O)Nc4ccc(C(*)=O)cc4)nn3)COCc3cn(CC(=O)Nc4ccc(C(*)=O)cc4)nn3)nn2)cc1 ; *C(=O)c1cccc(NC(=O)Cn2cc(COCC(COCc3cn(CC(=O)Nc4cccc(C(*)=O)c4)nn3)(COCc3cn(CC(=O)Nc4cccc(C(*)=O)c4)nn3)COCc3cn(CC(=O)Nc4cccc(C(*)=O)c4)nn3)nn2)c1]

In some embodiments of Formula (VIII), L is

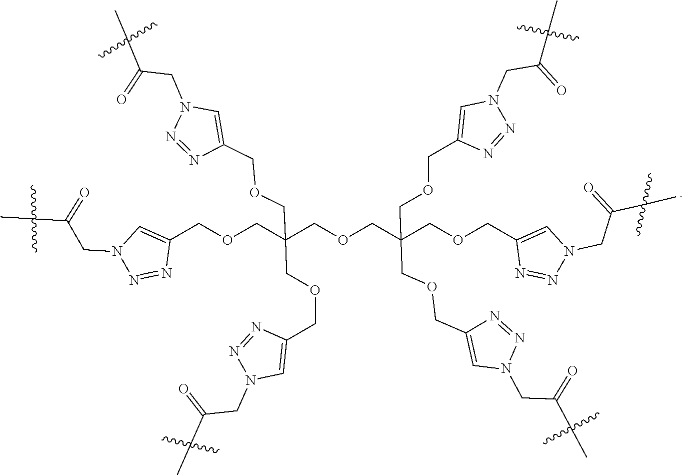 [SMILES: *C(=O)Cn1cc(COCC(COCc2cn(CC(*)=O)nn2)(COCc2cn(CC(*)=O)nn2)COCC(COCc2cn(CC(*)=O)nn2)(COCc2cn(CC(*)=O)nn2)COCc2cn(CC(*)=O)nn2)nn1]

In some embodiments of Formula (VIII), L is chosen from

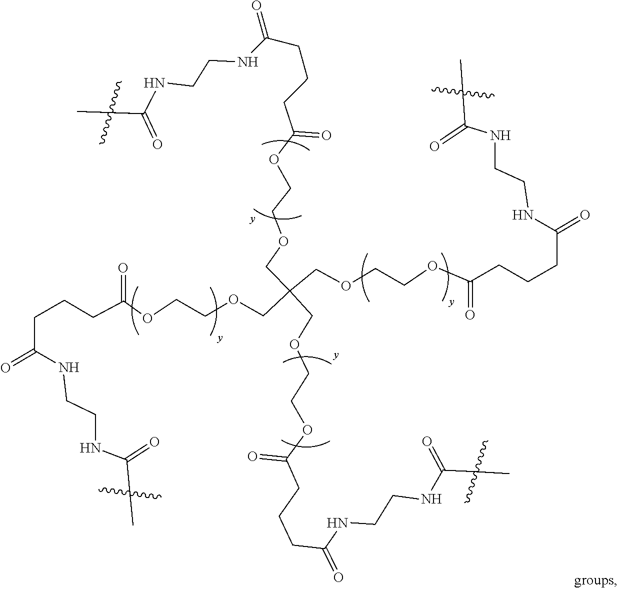

wherein each y, which may be identical or different, is independently chosen from integers ranging from 0 to 250.

In some embodiments of Formula (VIII), L is chosen from

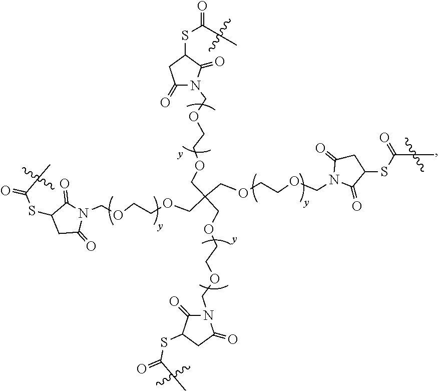

wherein each y, which may be identical or different, is independently chosen from integers ranging from 0 to 250.

In some embodiments of Formula (VIII), L is chosen from

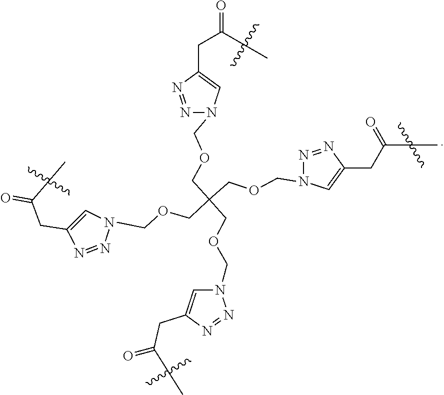 [SMILES: *C(=O)Cc1cn(COCC(COCn2cc(CC(*)=O)nn2)(COCn2cc(CC(*)=O)nn2)COCn2cc(CC(*)=O)nn2)nn1]

In some embodiments, at least one compound is chosen from compounds of Formula (VIII), wherein each R¹ is identical, each R² is identical, each R³ is identical, each R⁴ is identical, each R⁵ is identical, and each X is identical. In some embodiments, at least one compound is chosen from compounds of Formula (VII), wherein said compound is symmetrical.

Figures and examples illustrating the synthesis of compounds of Formula (VIII) are shown in PCT International Application Publication No. WO 2020/219417, which is incorporated by reference herein.

Also provided are pharmaceutical compositions comprising at least one compound chosen from compounds of Formula (I), (Ia), (II), (IIa), (III), (IIIa), (IV), (V), (IVa/Va), (IVb/Vb), (VI), (VII), and (VIII). These compounds and compositions may be used in the methods described herein. In some embodiments, provided are pharmaceutical compositions comprising at least one compound chosen from Compound A, Compound B, Compound C, Compound D, and Compound E. These compounds and compositions may be used in the methods described herein.

Also provided are pharmaceutical compositions comprising at least one pharmaceutically acceptable excipient and at least one compound chosen from compounds of Formula (T), (Ia), (II), (Ha), (III), (IIIa), (IV), (V), (IVa/Va), (IVb/Vb), (VI), (VII), and (VIII) and pharmaceutically acceptable salts of any of the foregoing. In some embodiments, provided are pharmaceutical compositions comprising at least one pharmaceutically acceptable excipient and at least one compound chosen from Compound A, Compound B, Compound C, Compound D, and Compound E, and pharmaceutically acceptable salts of any of the foregoing. These compounds and compositions may be used in the methods described herein.

In some embodiments, the at least one E-selectin antagonist is chosen from compounds of Formula (I), (Ia), (II), (IIa), (III), (IIIa), (IV), (V), (IVa/Va), (IVb/Vb), (VI), (VII), and (VIII). In some embodiments, the at least one E-selectin antagonist is chosen from compounds of Formula (I), (Ia), (II), (IIa), (III), (IIIa), (IV), (V), (IVa/Va), (IVb/Vb), (VI), (VII), and (VIII) and pharmaceutically acceptable salts of any of the foregoing.

In some embodiments, the at least one E-selectin antagonist is Compound A. In some embodiments, the at least one E-selectin antagonist is Compound B. In some embodiments, the at least one E-selectin antagonist is Compound C. In some embodiments, the at least one E-selectin antagonist is Compound D. In some embodiments, the at least one E-selectin antagonist is Compound E.

In some embodiments, the method further comprises administering at least one additional therapeutic agent. In some embodiments, the at least one additional therapeutic agent is chosen from antiviral and antiretroviral drugs. In some embodiments, the at least one additional therapeutic agent is molnupiravir. In some embodiments, the at least one additional therapeutic agent is chosen from anti-protozoal agents. In some embodiments, the at least one additional therapeutic agent is chosen from IL-6 inhibitors.

In some embodiments, the at least one additional therapeutic agent is remdesivir. In some embodiments, the at least one additional therapeutic agent is lopinavir. In some embodiments, the at least one additional therapeutic agent is ritonavir. In some embodiments, the at least one additional therapeutic agent is chloroquine. In some embodiments, the at least one additional therapeutic agent is hydroxychloroquine. In some embodiments, the at least one additional therapeutic agent is interferon-beta. In some embodiments, the at least one additional therapeutic agent is tocilizamib.

In some embodiments, the method further comprises administering at least one COVID-19 vaccine.

### EXAMPLE

The following example is intended to be illustrative and is not meant in any way to limit the scope of the disclosure.

### Prophetic Example 1: A Pilot Study to Assess the Safety, Tolerability and Efficacy of Selectin Inhibitor Compound a (GMI-1271) in Patients with COVID-19 Pneumonia

A Phase 1 prospective, interventional, single group assignment will be conducted at a single center (University of Michigan, Ann Arbor) in the United States to assess the safety, tolerability, and efficacy of Compound A in patients with COVID-19 pneumonia. Compound A is administered as an IV formulation that has been extensively tested in humans, including in healthy volunteers, patients with calf level deep vein thrombosis (DVT), and patients with acute myeloid leukemia (AML) and multiple myeloma (MM).

The primary objective of the open-label study is to evaluate if treatment with Compound A administered intravenously in addition to the best available therapy according to institutional guidelines is able to reduce the progression of acute respiratory failure, in patients with severe COVID-19 pneumonia. The primary endpoints include: (1) reduction in the progression to acute respiratory failure [Time Frame: 15 days for primary endpoint]; and (2) patients with a baseline PaO₂/FiO₂>=200: progression of respiratory failure is defined by: a. severe gas transfer deficit (PaO₂/FiO₂<200); b. persistent respiratory distress while receiving oxygen (persistent marked dyspnea, use of accessory respiratory muscles, paradoxical respiratory movements). The rate will be calculated as the proportion of patients who experienced at least one of the events above by day+15 from treatment start.

The secondary objectives include: (1) evaluating overall survival and all-cause mortality at day 15 and 28; (2) evaluating changes in the COVID ordinal outcomes scale; (3) assessing adverse events to evaluate the safety of Compound A; (4) assessing ventilator-free days, ICU-free days, oxygen, vasopressor free days; (5) evaluating changes in D-dimer; and (6) assessing the pharmacokinetics of Compound A used in this study. Exploratory objectives include: (1) examining the correlation of plasma soluble E-selectin concentrations with clinical outcomes; and (2) examining the correlations of other biomarkers of interest with clinical outcome.

15 subjects will be enrolled in the experimental cohort to receive Compound A, with parallel data collection on non-treated patients (concurrent control cohort n=15). Inclusion/exclusion criteria will be the same between the two groups. For subjects in the experimental cohort, Compound A (20 mg/kg BID, up to a maximum dose of 2500 mg) will be IV infused over 20 minutes on days 1-7. Protocol treatment must start within 48 hours of the admitted patient requiring supplemental oxygen for documented COVID-19 pneumonia. The concurrent control cohort, not treated with Compound A, will receive the best available therapy according to institutional guidelines.

Inclusion criteria include:
1. Adults, ages 18-75 years.
2. Willing and able to provide informed consent prior to performing study procedures unless they have a legally authorized representative (LAR).
3. Documented COVID-19 pneumonia: defined as upper respiratory tract specimen (nasopharyngeal swab (NPS) or viral throat swab) positive for COVID-19 and imaging (CXR/CT scan) suggestive of COVID-19 pneumonia.
4. Confirmed coronavirus (SARS-CoV-2) infection, enrolled S 48 hours of beginning oxygen need for COVID-19+ confirmed hospital admission.
5. Currently hospitalized requiring supplemental oxygen.
6. Have severe COVID-19 according to the World Health Organization (WHO) Interim Guidance with confirmation by real-time RT-PCR assay. The enrollment criteria with one of the following: respiratory distress, respiratory rate (RR) ≥30 beats/min; oxygen saturation level less than 93% in resting state; or partial pressure of oxygen (PaO₂)/oxygen concentration (FiO₂) ≤300 mmHg.
7. WHO, Clinical management of severe acute respiratory infection when Novel coronavirus (nCoV) infection is suspected: Interim guidance. https://www. who.int/publications-detail/clinical-management-of-severe-acute-respiratory-infectionwhen-novel-coronavirus-(ncov)-infection-is-suspected.
8. Willing and able to participate in all required evaluations and procedures.

Exclusion criteria include:
1. In the opinion of at least two investigators, unlikely to survive for >48 hours from screening.
2. Severe chronic respiratory disease (e.g. COPD or other) requiring supplemental oxygen and/or having required mechanical ventilation pre-COVID-19 infection.
3. Concurrent enrollment in a COVID-related interventional drug trial. Use of remdesivir, steroids, and convalescent plasma are permitted along with other standard of care therapies for COVID.
4. Currently on invasive mechanical ventilation.
5. Hypotension defined as systolic blood pressure <90 mmHg on two sequential readings at least 4 hours apart.
6. Total Bilirubin ≤3× upper limit of normal (ULN), Creatinine Clearance ≥30 mL/min/1.73 m².
7. Pregnant or breastfeeding.
8. Known diagnosis of an acute thrombosis on admission.
9. Concurrent dual antithrombotic therapy (aspirin or P2Y12 inhibitor plus anticoagulation to treat deep venous thrombosis or pulmonary embolism (single antiplatelet or anticoagulant agent at prophylactic dose is permitted).
10. Concomitant use of thrombolytic therapy.
11. Concomitant therapeutic systemic anticoagulant therapy (e.g. heparin, warfarin, direct thrombin inhibitors and direct factor Xa inhibitors).
12. History of recent major bleeding, defined in accordance with the criteria of the International Society on Thrombosis and Hemostasis (ISTH).
13. History of bleeding disorder thought to impose excessive bleeding risk, as per investigator discretion.
14. Hemodynamic instability, defined as inability to maintain mean arterial pressure.
15. Hypersensitivity to the active substance or to any of the excipients of Compound A.
16. Any physical examination findings and/or history of any illness that, in the opinion of the investigator, might confound the results of the study or pose an additional risk to the patient by their participation in the study.

### REFERENCES

The following references are hereby incorporated by reference in their respective entireties:
1. Huang C. et al 2020 Clinical features of patients infected with 2019 novel coronavirus in Wuhan, China. Lancet https://doi.org/10.1016/s0140-6736(20)30183-5.
2. Potey P., Rossi A. G., Lucas C. D. and Dorward D. A. Neutrophils in the initiation and resolution of acute pulmonary inflammation: understanding biological function and therapeutic potential. J. Pathol. 247:672-685 (2019).
3. Lyall F., Boswell F., Young A., Clark C. J., and Greer I. A. The cytokine interleukin-6 increases expression of the cell adhesion molecules E-selectin and VCAM-1 on endothelial cells in vitro: a role in preclampsia? Hyper in Preg 16(3):403-415 (1997).
4. Vora M., Romero L. I. and Karasek M. A. Interleukin-10 induces E-selectin on small and large blood vessel endothelial cells. J. Exp. Med. 184:821-829 (1996).
5. Xu Z. et.al. Predictive value of combined LIPS and ANG-2 level in critically III patients with ARDS risk factors. Mediators Inflamm (2018).
6. Osaka D. et.al. Soluble endothelial selectin in acute lung injury complicated by severe pneumonia. Int. J. Med. Sci. 8(4):302-308 (2011).
7. Okajima K., Harada N., Sakurai G., Soga Y., Suga H., Terada T. and Nakagawa T. Rapid assay for plasma soluble E-selectin predicts the development of acute respiratory distress syndrome in patients with systemic inflammatory response syndrome. Translational Res. 148:295-300 (2006).
8. Meyer N. J. et.al. Myeloperoxidase-derived 2-chlorofatty acids contribute to human sepsis mortality via acute respiratory distress syndrome. JCI insight 2(23) (2017).
9. Ruchaud-Sparagano M-H., Dorst E. M., Donnelly S. C., Bird M. I., Haslett C. and Dransfield I. Potential pro-inflammatory effects of soluble E-selectin upon neutrophil function. Eur. J. Immunol. 28:80-89 (1998).
10. Mulligan M. S., Lowe J. B., Larsen R. D., Paulson J., Zheng Z., DeFrees S., Maemura K., Fukuda M. and Ward P. A. Protective effects of sialylated oligosaccharides in immune complex-induced acute lung injury. J. Exp. Med. 179:623-631 (1993).
11. Wun T., et. al. Pan-selectin antagonist Rivipansel (GMI-1070) reduces soluble E-selectin levels while improving clinical outcomes in SCD vaso-occlusive crisis. Blood 124(21):2704 (2014).
12. DeAngelo D. J., Liesveld J. L., Jonas B. A., O'Dwyer M. E., Bixby D. L., Magnani J. L., Thackray H. M. and Becker P. S. A phase U/l study of GMI-1271, a novel E-selectin antagonist, in combination with induction chemotherapy in relapsed/refractory and previously untreated elderly patients with acute myeloid leukemia; results to date. Blood 128(22):4049 (2016).

1. A method of treating COVID-19 in a patient in need thereof, the method comprising administering to the patient an effective amount of at least one E-selectin antagonist or a pharmaceutical composition comprising an effective amount of at least one E-selectin antagonist.

2. A method of treating a disease, disorder, and/or condition associated with COVID-19 in a patient in need thereof, the method comprising administering to the patient an effective amount of at least one E-selectin antagonist or a pharmaceutical composition comprising an effective amount of at least one E-selectin antagonist.

3. The method according to claim 2, wherein the disease, disorder, and/or condition associated with COVID-19 is acute lung injury.

4. The method according to claim 2, wherein the disease, disorder, and/or condition associated with COVID-19 is acute respiratory distress syndrome.

5. The method according to claim 2, wherein the disease, disorder, and/or condition associated with COVID-19 is pneumonia.

6. The method according to claim 2, wherein the disease, disorder, and/or condition associated with COVID-19 is ARDS pneumonia.

7. A method of treating acute respiratory distress syndrome in a patient in need thereof, the method comprising administering to the patient an effective amount of at least one E-selectin antagonist or a pharmaceutical composition comprising an effective amount of at least one E-selectin antagonist.

8. A method of treating pneumonia in a patient in need thereof, the method comprising administering to the patient an effective amount of at least one E-selectin antagonist or a pharmaceutical composition comprising an effective amount of at least one E-selectin antagonist.

9. The method according to claim 2, wherein the at least one E-selectin antagonist is chosen from 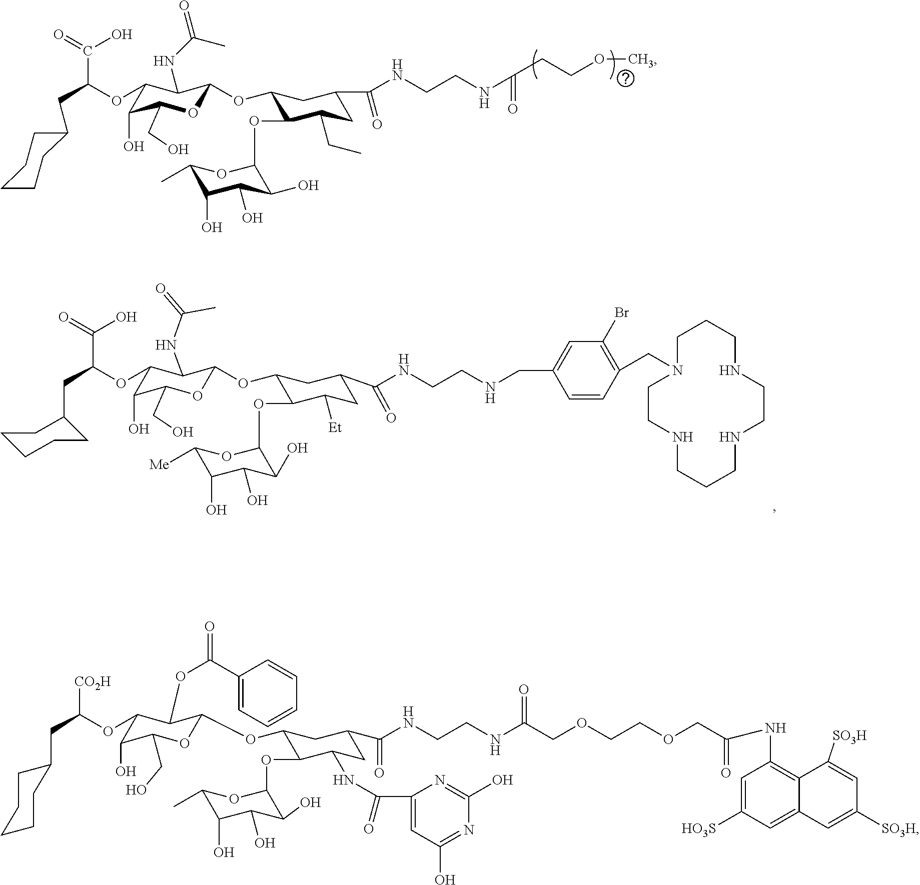 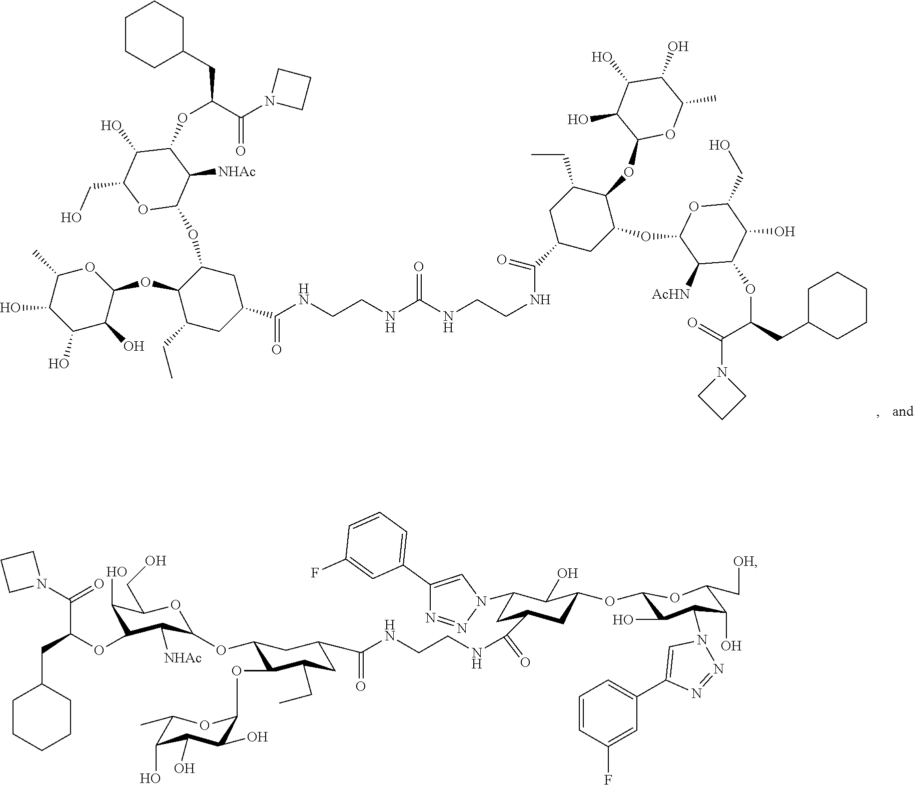
and pharmaceutically acceptable salts of any of the foregoing.

10. The method according to claim 2, wherein the at least one E-selectin antagonist is chosen from compounds of Formula (I): 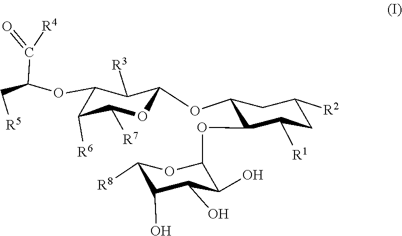
isomers of Formula (I), tautomers of Formula (I), and pharmaceutically acceptable salts of any of the foregoing, wherein:
R¹ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R² is chosen from H, -M, and -L-M;
R³ is chosen from —OH, —NH₂, —OC(═O)Y¹, —NHC(═O)Y¹, and —NHC(═O)NHY¹ groups, wherein Y¹ is chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups;
R⁴ is chosen from —OH and —NZ¹Z² groups, wherein Z¹ and Z², which may be identical or different, are each independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Z¹ and Z² may together form a ring;
R⁵ is chosen from C₃₋₈ cycloalkyl groups;
R⁶ is chosen from —OH, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R⁷ is chosen from —CH₂OH, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R⁸ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
L is chosen from linker groups; and
M is a non-glycomimetic moiety chosen from polyethylene glycol, thiazolyl, chromenyl, —C(═O)NH(CH₂)₁₋₄NH₂, C₁₋₈ alkyl, and —C(═O)OY groups, wherein Y is chosen from C₁₋₄ alkyl, C₂₋₄ alkenyl, and C₂₋₄ alkynyl groups.

11. The method according to claim 10, wherein the at least one E-selectin antagonist is chosen from compounds of Formula (I), wherein the non-glycomimetic moiety comprises polyethylene glycol.

12. The method according to claim 10 or 11, wherein L is —C(═O)NH(CH₂)₁₋₄NHC(═O)—.

13. The method according to claim 2, wherein the at least one E-selectin antagonist is chosen from compounds of Formula (Ia): 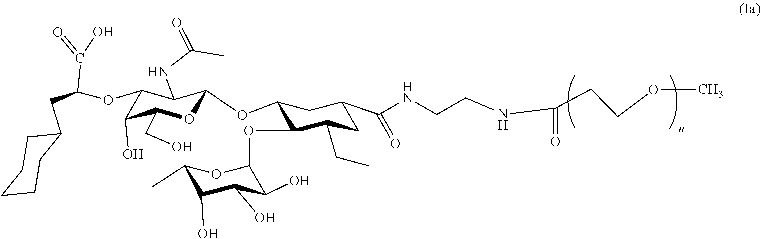
and pharmaceutically acceptable salts thereof, wherein n is chosen from integers ranging from 1 to 100.

14. The method according to claim 13, wherein n is chosen from 4, 8, 12, 16, 20, 24, and 28.

15. The method according to claim 2, wherein the at least one E-selectin antagonist is chosen from Compound A: 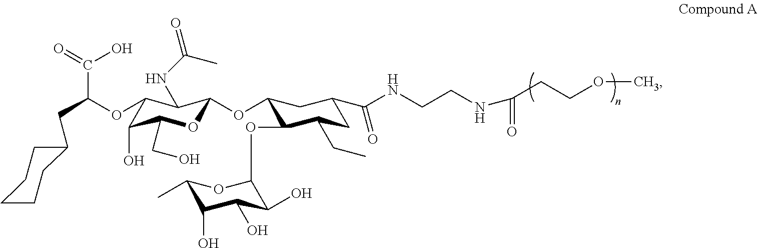
and pharmaceutically acceptable salts thereof.

16. The method according to claim 2, wherein the at least one E-selectin antagonist is a heterobifunctional antagonist chosen from compounds of Formula (II): 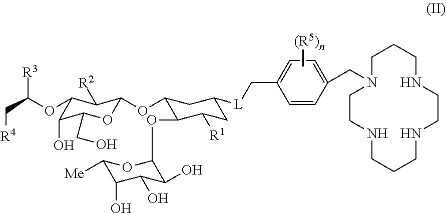
isomers of Formula (II), tautomers of Formula (II), and pharmaceutically acceptable salts of any of the foregoing, wherein:
R¹ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R² is chosen from —OH, —NH₂, —OC(═O)Y¹, —NHC(═O)Y¹, and —NHC(═O)NHY¹ groups, wherein Y¹ is chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups;
R³ is chosen from —CN, —CH₂CN, and —C(═O)Y² groups, wherein Y² is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Z¹ and Z² may together form a ring;
R⁴ is chosen from C₃₋₈ cycloalkyl groups;
R⁵ is independently chosen from H, halo, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
n is chosen from integers ranging from 1 to 4; and
L is chosen from linker groups.

17. The method according to claim 16, wherein the at least one E-selectin antagonist is a heterobifunctional antagonist chosen from compounds of Formula (IIa): 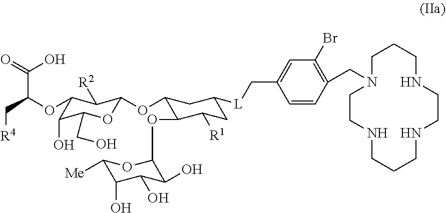
and pharmaceutically acceptable salts thereof.

18. The method according to claim 2, wherein the at least one E-selectin antagonist is a heterobifunctional antagonist chosen from Compound B: 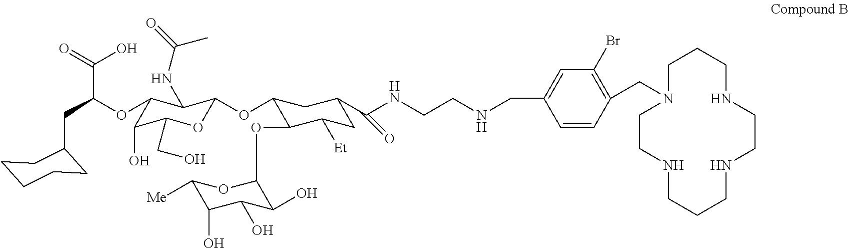 [SMILES: CCC1CC(C(=O)NCCNCc2ccc(CN3CCCNCCNCCCNCC3)c(Br)c2)CC(OC2OC(CO)C(O)C(O[C@@H](CC3CCCCC3)C(=O)O)C2NC(C)=O)C1OC1OC(C)C(O)C(O)C1O]
and pharmaceutically acceptable salts thereof.

19. The method according to claim 2, wherein the at least one E-selectin antagonist is a heterobifunctional pan-selectin antagonist chosen from compounds of Formula (III): 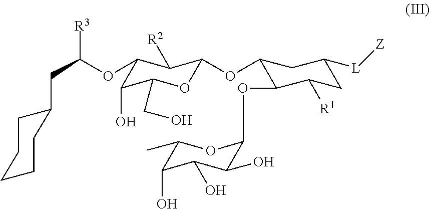
isomers of Formula (III), tautomers of Formula (III), and pharmaceutically acceptable salts of any of the foregoing, wherein:
R¹ is chosen from H, C₁₋₈ alkyl, C₂₋₈ alkenyl, C₂₋₈ alkynyl, C₄₋₁₆ cycloalkylalkyl 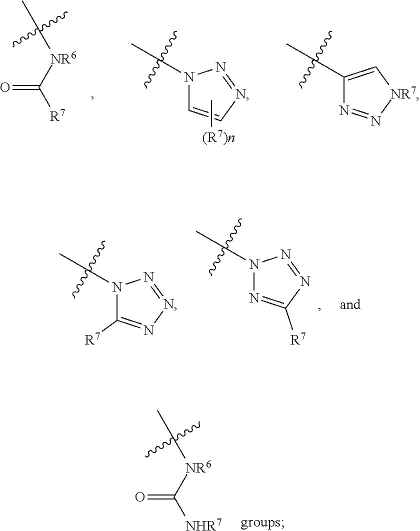
R² is chosen from C₁₋₈ alkyl, C₂₋₈ alkenyl, C₂₋₈ alkynyl, C₄₋₁₆ cycloalkylalkyl, —OH, —OX¹, halo, —NH₂, —OC(═O)X¹, —NHC(═O)X¹, and —NHC(═O)NHX¹ groups, wherein X¹ is chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups;
R³ is chosen from —CN, —CH₂CN, and —C(═O)X² groups, wherein X² is chosen from C₁₋₈ alkyl, C₂₋₈ alkenyl, C₂₋₈ alkynyl, —OY², —NHOH, —NHOCH₃, —NHCN, and —NY²Y³ groups, wherein Y² and Y³, which may be identical or different, are independently chosen from H, C₁₋₈ alkyl, C₂₋₈ alkenyl, C₂₋₈ alkynyl, and C₄₋₁₆ cycloalkylalkyl groups, wherein Y² and Y³ may join together to form a ring;
R⁶ is chosen from H, C₁₋₈ alkyl, C₂₋₈ alkenyl, C₂₋₈ alkynyl, C₄₋₁₆ cycloalkylalkyl, and —C(═O)R⁷ groups;
each R⁷ is independently chosen from H, C₁₋₈ alkyl, C₂₋₈ alkenyl, C₂₋₈ alkynyl, C₄₋₁₆ cycloalkylalkyl, 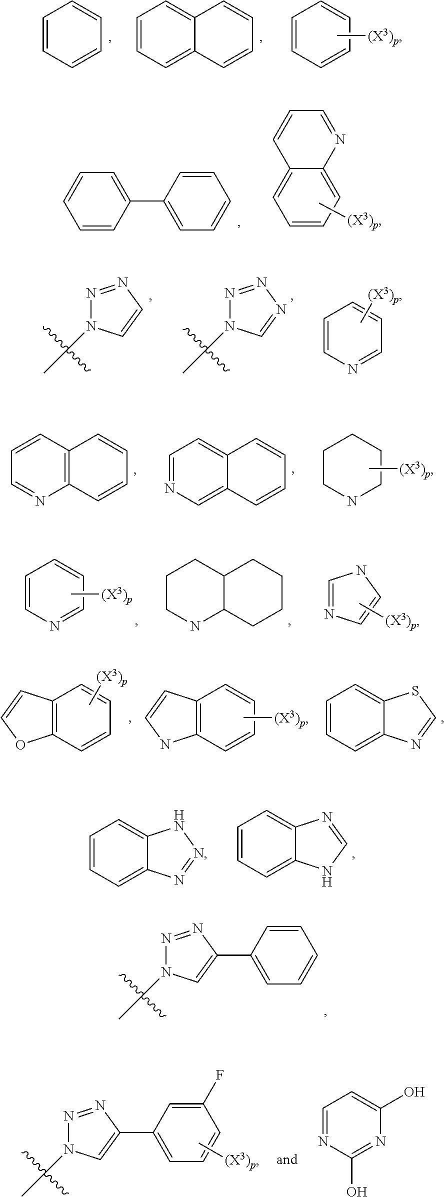
groups, wherein each X³ is independently chosen from H, —OH, Cl, F, N₃, —NH₂, C₁₋₈ alkyl, C₂₋₈ alkenyl, C₂₋₈ alkynyl, C₆₋₁₄ aryl, —OC₁₋₈ alkyl, —OC₂₋₈ alkenyl, —OC₂₋₈ alkynyl, and —OC₆₋₁₄ aryl groups, wherein any of the above ring compounds may be substituted with one to three groups independently chosen from Cl, F, C₁₋₈ alkyl, C₂₋₈ alkenyl, C₂₋₈ alkynyl, C₆₋₁₄ aryl, and —OY⁴ groups, wherein Y⁴ is chosen from H, C₁₋₈ alkyl, C₂₋₈ alkenyl, C₂₋₈ alkynyl, and C₆₋₁₄ aryl groups;
n is chosen from integers ranging from 0 to 2;
p is chosen from integers ranging from 0 to 3;
L is chosen from linker groups; and
Z is chosen from benzyl amino sulfonic acid groups.

20. The method according to claim 19, wherein the at least one E-selectin antagonist is chosen from Formula (IIIa): 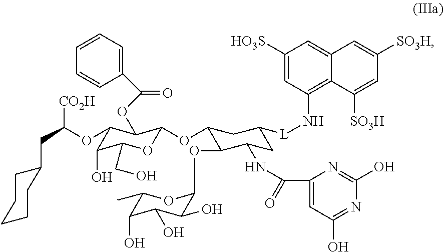
tautomers of Formula (IIIa), and pharmaceutically acceptable salts of any of the foregoing.

21. The method according to claim 2, wherein the at least one E-selectin antagonist is a heterobifunctional pan-selectin antagonist chosen from Compound C: 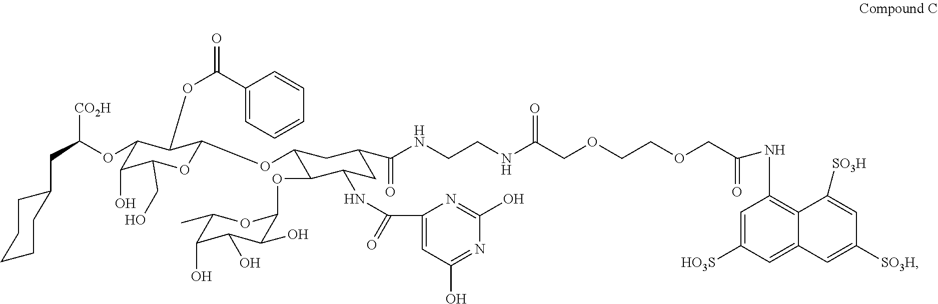 [SMILES: CC1OC(OC2C(NC(=O)c3cc(O)nc(O)n3)CC(C(=O)NCCNC(=O)COCCOCC(=O)Nc3cc(S(=O)(=O)O)cc4cc(S(=O)(=O)O)cc(S(=O)(=O)O)c34)CC2OC2OC(CO)C(O)C(O[C@@H](CC3CCCCC3)C(=O)O)C2OC(=O)c2ccccc2)C(O)C(O)C1O]
tautomers of Compound C, and pharmaceutically acceptable salts of any of the foregoing.

22. The method according to claim 2, wherein the at least one E-selectin antagonist is chosen from compounds of Formula (IV): 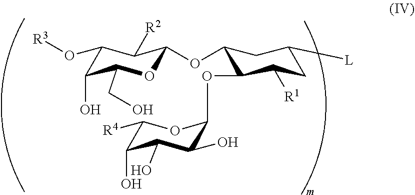
prodrugs of Formula (IV), isomers of Formula (IV), tautomers of Formula (IV), and pharmaceutically acceptable salts of any of the foregoing, wherein
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NY¹Y² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;
each R³, which may be identical or different, is independently chosen from 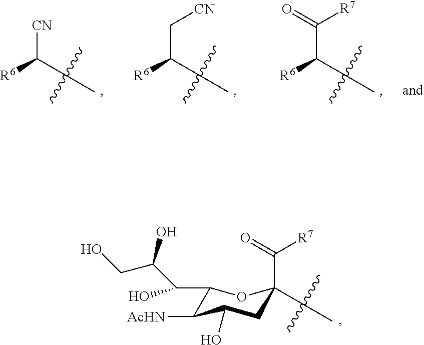
wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is chosen from integers ranging from 2 to 256; and
L is chosen from linker groups.

23. The method according to claim 2, wherein the at least one E-selectin antagonist is chosen from compounds of Formula (V): 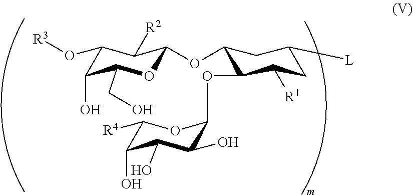
prodrugs of Formula (V), isomers of Formula (V), tautomers of Formula (V), and pharmaceutically acceptable salts of any of the foregoing, wherein
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NY¹Y² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;
each R³, which may be identical or different, is independently chosen from 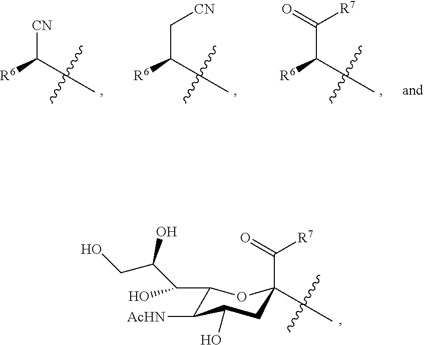
wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is 2; and
L is chosen from 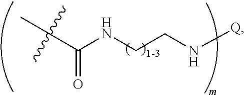
wherein Q is a chosen from 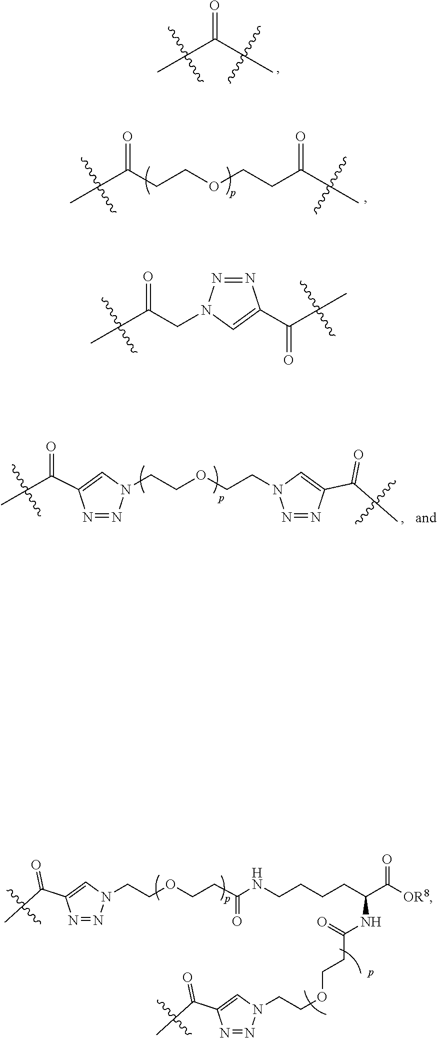
wherein R⁸ is chosen from H, C₁₋₈ alkyl, C₆₋₁₈ aryl, C₇₋₁₉ arylalkyl, and C₁₋₁₃ heteroaryl groups and each p, which may be identical or different, is independently chosen from integers ranging from 0 to 250.

24. The method according to claim 2, wherein the at least one E-selectin antagonist is chosen from Compound D:

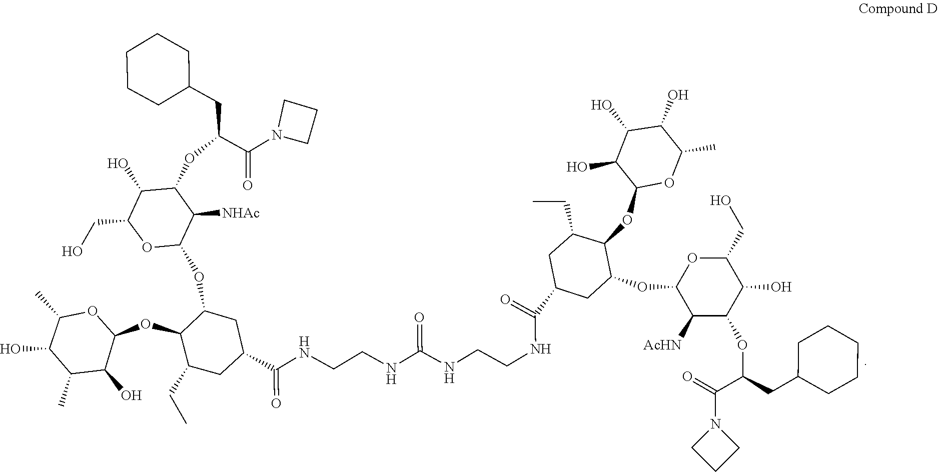

25. The method according to claim 2, wherein the at least one E-selectin antagonist is chosen from Compound E:

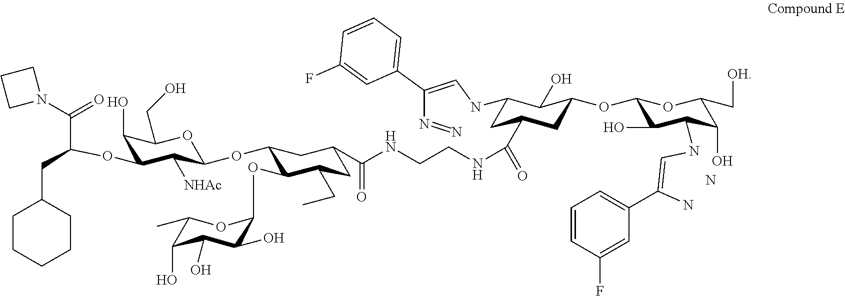

26. The method according to claim 2, further comprising administering at least one additional therapeutic agent.

27. The method according to claim 26, wherein the at least one additional therapeutic agent is chosen from antiviral and antiretroviral drugs.

28. The method according to claim 26, wherein the at least one additional therapeutic agent is molnupiravir.

29. The method according to claim 26, wherein the at least one additional therapeutic agent is chosen from anti-protozoal agents.

30. The method according to claim 26, wherein the at least one additional therapeutic agent is chosen from IL-6 inhibitors.

31. The method according to claim 2, further comprising administering at least one COVID-19 vaccine.
